(19) United States
(12) Patent Application Publication
(10) Pub. No.: US 2026/0015419 A1
(43) Pub. Date: Jan. 15, 2026

(54) COMBINATION OF MACROPHAGE-DIRECTED IMMUNOTHERAPY AND CYTOKINES FOR TREATMENT OF CANCER

(71) Applicants: Whitehead Institute for Biomedical Research, Cambridge, MA (US); Dana-Farber Cancer Institute, Inc., Boston, MA (US)

(72) Inventors: Kipp Weiskopf, Wellesley, MA (US); Asaf Maoz, Chestnut Hill, MA (US)

(73) Assignees: Whitehead Institute for Biomedical Research, Cambridge, MA (US); Dana-Farber Cancer Institute, Inc., Boston, MA (US)

(21) Appl. No.: 18/994,346

(22) PCT Filed: Jul. 14, 2023
(86) PCT No.: PCT/US2023/027749

§ 371 Date: Jan. 14, 2025

Related U.S. Application Data
(60) Provisional application No. 63/389,626, filed on Jul. 15, 2022.

(51) Int. Cl. C07K 16/28; A61K 38/00; A61P 35/00; C07K 14/54; C07K 14/555

(52) U.S. Cl. CPC C07K 16/2803; A61P 35/00; C07K 14/5428; C07K 14/555; A61K 38/00; C07K 2319/00

(57) ABSTRACT

The present disclosure provides methods and compositions related to the combination therapy of a macrophage-directed immunotherapy and a cytokine (e.g., IL-10). The combination of the macrophage-directed immunotherapy and a cytokine is useful in treating and/or preventing cancer (e.g., lung cancer) in a subject. Therapies that activate macrophages are emerging in cancer immunotherapy. One potential therapeutic target is the CD47-SIRPa, interaction, which acts as a myeloid immune checkpoint. Cluster of Differentiation 47 (CD47) is highly expressed on many different types of cancer cells, including lung cancer cells. CD47 binds to an inhibitory receptor, signal regulatory protein alpha (SIRPa,), that is expressed on the surface of macrophages and other myeloid immune cells.

### RELATED APPLICATIONS

This patent application claims priority under 35 U.S.C. § 119(e) to U.S. Provisional Application U.S. Ser. No. 63/389,626, filed Jul. 15, 2022. The entire content of each is incorporated herein by reference.

### BACKGROUND

Therapies that activate macrophages are emerging in cancer immunotherapy. One potential therapeutic target is the CD47-SIRPα interaction, which acts as a myeloid immune checkpoint. Cluster of Differentiation 47 (CD47) is highly expressed on many different types of cancer cells, including lung cancer cells. CD47 binds to an inhibitory receptor, signal-regulatory protein alpha (SIRPα), that is expressed on the surface of macrophages and other myeloid immune cells. When CD47 binds to SIRPα, it sends inhibitory signals to macrophages that prevent phagocytosis of the cancer cell. CD47-blocking therapies stimulate macrophage phagocytosis of cancer cells and are effective across many preclinical cancer models. They have demonstrated efficacy in clinical trials for relapsed/refractory lymphoma and are under investigation for other solid and hematologic malignancies. But, there exists a need for improving and enhancing macrophage-mediated cancer immunotherapy.

Cytokines are a broad category of small proteins (ca. 5-25 kDa) important in cell signaling. Cytokines are involved in autocrine, paracrine, and endocrine signaling as immunomodulating agents. Some have been investigated in cancer treatment. Interleukin 10 (IL-10) is a cytokine with both pro-inflammatory and anti-inflammatory characteristics having multiple pleiotropic effects in immunoregulation and inflammation. However, studies have shown that IL-10 has an inhibitory effect on macrophages but possibly activate cytotoxic T-cells, and the majority of cytokines used in pre-clinical and clinical cancer immunotherapy is focused on T-cell mediated cancer immunotherapies.

### SUMMARY

The present disclosure stems from the discovery that cytokines unexpectedly potentiate the effects of macrophage-mediated cancer immunotherapy. Thus, combining cytokines (e.g., IL-10) or agents that act on cytokine receptors (e.g., an agent having an agonistic effect on a cytokine such as IL-10) with immunotherapies that stimulate macrophages (e.g., CD47-blocking antibodies) provides effective therapies for treating cancer. Accordingly, the present disclosure provides new combination regimens comprising macrophage-directed immunotherapies and cytokines (e.g., IL-10) for treating cancer patients.

In one aspect, the present disclosure provides methods of treating a proliferative disease in a subject in need thereof, the method comprising administering a macrophage-directed immunotherapy and a cytokine, or a modification, fragment, or variant thereof.

In another aspect, the present disclosure provides methods of treating a proliferative disease in a subject in need thereof, the method comprising administering a macrophage-directed immunotherapy and an agent that acts on a cytokine receptor. In certain embodiments, the agent that acts on a cytokine receptor is an agonist of the cytokine receptor (e.g., IL-10 receptor). In certain embodiments, the agent that acts on a cytokine receptor has an agonistic effect on the cytokine receptor (e.g., IL-10 receptor).

In certain embodiments, the macrophage-directed immunotherapy is a macrophage immune checkpoint inhibitor. In certain embodiments, the macrophage-directed immunotherapy comprises modulation of CD47, SIRPα, EGFR, MHC I, CD24, CALR, CD40, PD-L1, APMAP, GPR84, VCAM1, CD11b, SIGLEC-10, PD-L2, PD-1, CD73, epCAM, Galectin-9, CD14, CD80, CD86, SIRPb, SIRPg, SLAMF7, MARCO, AXL, CLEVER-1, ILT4, TIM-3, TIM-4, LRP-1, calreticulin, TREM1, TREM2, GD2, FcgRI, FcgRIIa, FcgRIIb, FcgRIII, MUC1, CD44, CD63, CD36, CD84, CD164, CD82, CD18, SIGLEC-7, CD166, CD39, CD46, LILRA1, LILRA2(ILT1), LILRA3 (ILT6), LILRA4 (ILT7), LILRB1 (ILT2), LILRB2(ILT4), LILRB3 (ILT5), LILRB4 (ILT3), LILRB5, CD85b (ILT8 or ILT9), CD85m (ILT10), CD85f (ILT11), CD276, CD88, CD99, PILRa, Siglec-9, CD206, CD163, CD84 (SLAMF5), C3aR, or CLEC12A. In certain embodiments, the macrophage-directed immunotherapy comprises modulation of CD47, SIRPα, EGFR, CD40, PD-L1, PD-L2, CD73, or EpCAM.

In certain embodiments, the macrophage-directed immunotherapy is an anti-CD47 antibody, an anti-SIRPα antibody, a SIRPα-Fc fusion protein, an anti-EGFR antibody, an anti-CD40 antibody, an anti-PD-L1 antibody, an anti-PD-L2 antibody, an anti-CD73 antibody, or an anti-EpCAM antibody. In certain embodiments, the macrophage-directed immunotherapy is an anti-CD47 antibody.

In certain embodiments, the cytokine is an anti-inflammatory cytokine, or a modification, fragment, or variant thereof. In certain embodiments, the cytokine is a class II cytokine, or a modification, fragment, or variant thereof.

In certain embodiments, the cytokine is interleukin-10 (IL-10), interleukin-19 (IL-19), interleukin-20 (IL-20), interleukin-22 (IL-22), interleukin-24 (IL-24), interleukin-26 (IL-26), interleukin-28A (IL-28A), interleukin-28B (IL-28B), interleukin-29 (IL-29), a type-I interferon, a type-II interferon, or a modification, fragment, or variant thereof. In certain embodiments, the cytokine is interleukin-10 (IL-10), interleukin-19 (IL-19), interleukin-20 (IL-20), interleukin-22 (IL-22), interleukin-24 (IL-24), interleukin-26 (IL-26), interleukin-28A (IL-28A), interleukin-28B (IL-28B), or interleukin-29 (IL-29), or a modification, fragment, or variant thereof. In certain embodiments, the cytokine is interleukin-10 (IL-10), or a modification, fragment, or variant thereof. In certain embodiments, the cytokine is a variant of interleukin-10 (IL-10) having at least 80%, 85%, 90%, 95%, 97%, 98%, 99%, or 99.5% sequence identity to the amino acid sequence of IL-10. In certain embodiments, the cytokine is a modified version of interleukin-10 (IL-10). In certain embodiments, the cytokine is pegilodecakin.

In certain embodiments, the cytokine is a type-I interferon (e.g., interferon alpha (IFN-α), interferon omega (IFN-ω)) or a type-II interferon (e.g., interferon gamma (IFN-γ)), or a modification, fragment, or variant thereof.

In certain embodiments, the proliferative disease is cancer. In certain embodiments, the cancer is bladder cancer, cervical cancer, dermatofibrosarcoma protuberans, endocrine tumors, neuroendocrine tumors, neuroblastoma, anaplastic large cell lymphoma, glioblastoma multiforme, bile duct cancer, stomach cancer, colon cancer, rectal cancer, melanoma, colorectal cancer, brain cancer, head and neck cancer, thyroid cancer, soft tissue cancer, lung cancer, colon cancer, kidney cancer, liver cancer, gastric cancer, gastrointestinal stromal tumor, giant cell tumor, esophageal cancer, gastroesophageal cancer, breast cancer, ovarian cancer, prostate cancer, endometrial cancer, pancreatic cancer, leukemia, lymphoma, multiple myeloma, colon adenocarcinoma, lung adenocarcinoma, cutaneous melanoma, gastrointestinal cancer, anal cancer, glioblastoma, epithelian tumors of the head and neck, laryngeal cancer, oral cancer, myelodysplastic disorders, myeloproliferative disorders, ovarian epithelial cancer, fallopian tube cancer, primary peritoneal cancer, plexiform neurofibroma, skin cancer, soft tissue sarcoma, solid tumors with an NTRK gene fusion, or systemic mastocytosis.

In certain embodiments, the cancer is colorectal cancer. In certain embodiments, the cancer is colon cancer. In certain embodiments, the cancer is ovarian cancer. In certain embodiments, the cancer is lung cancer (e.g., non-small cell lung cancer).

In another aspect, disclosed is a pharmaceutical composition comprising a macrophage-directed immunotherapy and a cytokine, and optionally a pharmaceutically acceptable excipient.

In another aspect, disclosed is a kit comprising a macrophage-directed immunotherapy and a cytokine and instructions for using the kit.

In another aspect, disclosed is a bifunctional compound, or a pharmaceutically acceptable salt thereof, comprising a macrophage-directed immunotherapy and a cytokine, or a modification, fragment, or variant thereof.

### Definitions

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃)(e.g., n-propyl, isopropyl), butyl (C₄)(e.g., n-butyl, tert-butyl, sec-butyl, iso-butyl), pentyl (C₅)(e.g., n-pentyl, 3-pentanyl, amyl, neopentyl, 3-methyl-2-butanyl, tertiary amyl), and hexyl (C₆)(e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

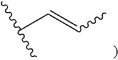

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds)(“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂-6 alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂-8 alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), bicyclo[6.1.0]non-4-enyl (C₉), bicyclo[6.1.0]nonanyl (C₉), bicyclo[6.1.0]non-4-ynyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic)₄n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic)₄n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR)₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR)₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH) OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(RC)₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(RC)₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined herein.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl) methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl (pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(RC)₃⁺X⁻, —P(OR)₂, —P(OR)₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present disclosure.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “fusion protein” or “chimeric protein” is a protein created through the joining of two or more genes that originally coded for separate proteins. Translation of this fusion gene results in a single or multiple polypeptides with functional properties derived from each of the original proteins.

The term “inhibit” or “inhibition” in the context of modulating level (e.g., expression and/or activity) of a target is not limited to only total inhibition. Thus, in some embodiments, partial inhibition or relative reduction is included within the scope of the term “inhibition.” In some embodiments, the term refers to a reduction of the level (e.g., expression, and/or activity) of a target to a level that is reproducibly and/or statistically significantly lower than an initial or other appropriate reference level, which may, for example, be a baseline level of a target. In some embodiments, the term refers to a reduction of the level (e.g., expression and/or activity) of a target to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of a target.

As used herein, the term “inhibitor” refers to an agent whose presence or level correlates with decreased level or activity of a target to be modulated. In some embodiments, an inhibitor may act directly (in which case it exerts its influence directly upon its target, for example by binding to the target); in some embodiments, an inhibitor may act indirectly (in which case it exerts its influence by interacting with and/or otherwise altering a regulator of a target, so that level and/or activity of the target is reduced). In some embodiments, an inhibitor is one whose presence or level correlates with a target level or activity that is reduced relative to a particular reference level or activity (e.g., that observed under appropriate reference conditions, such as presence of a known inhibitor, or absence of the inhibitor as disclosed herein, etc.).

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. A “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The terms “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount effective to prevent a condition, or one or more symptoms associated with the condition and/or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, hematological malignancies. The term “hematological malignancy” refers to tumors that affect blood, bone marrow, and/or lymph nodes. Exemplary hematological malignancies include, but are not limited to, leukemia, such as acute lymphocytic leukemia (ALL)(e.g., B-cell ALL, T-cell ALL), acute myelocytic leukemia (AML)(e.g., B-cell AML, T-cell AML), chronic myelocytic leukemia (CML)(e.g., B-cell CML, T-cell CML), and chronic lymphocytic leukemia (CLL)(e.g., B-cell CLL, T-cell CLL)); lymphoma, such as Hodgkin lymphoma (HL)(e.g., B-cell HL, T-cell HL) and non-Hodgkin lymphoma (NHL)(e.g., B-cell NHL, such as diffuse large cell lymphoma (DLCL)(e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt lymphoma, Waldenström's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL)(e.g., cutaneous T-cell lymphoma (CTCL)(e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; and multiple myeloma (MM). Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD)(e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “immunotherapy” refers to a treatment of disease by inducing, enhancing, or suppressing an immune response. Immunotherapies designed to elicit or amplify an immune response are classified as activation immunotherapies, while immunotherapies that reduce or suppress an immune response are classified as suppression immunotherapies. Immunotherapy may encompass treatment with a molecular entity (e.g., immunotherapeutic agent) and/or a non-molecular entity (e.g., adoptive cell transfer).

The term “macrophage-directed immunotherapy” refers to an immunotherapy that activates macrophages, and it derives its therapeutic effect by stimulating macrophages. Such stimulation can mobilize macrophage and myeloid components to destroy a tumor and its stroma, including the tumor vasculature. Macrophages can be induced to secrete antitumor cytokines and/or to perform phagocytosis, including antibody-dependent cellular phagocytosis.

The term “immunotherapeutic agent” refers to a molecular entity that induces, enhances, or suppresses an immune response. Immunotherapeutic agents include, but are not limited to, monoclonal antibodies, cytokines, chemokines, vaccines, small molecule inhibitors, and small molecule agonists.

The term “immune checkpoint inhibitor” refers to an agent that blocks certain proteins made by some types of immune system cells (e.g., T cells, macrophages) and some cancer cells. These proteins function to keep immune responses in check and can also function to keep immune system cells (e.g., T cells, macrophages) from killing cancer cells. When these proteins are blocked, immune system function is restored and the immune system is released enabling the desired immune system cells to kill cancer cells. Some immune checkpoint inhibitors are useful in treating cancer. A “macrophage immune checkpoint inhibitor” functions to stimulate macrophage phagocytosis of cancer cells. For example, CD47 is associated with a macrophage immune checkpoint (CD47/SIRPα as described herein). CD47-blocking therapies thus stimulate macrophage phagocytosis of cancer cells and are effective in treating cancer.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities such as cells and tissues. Biologics may be isolated from a variety of natural sources (e.g., human, animal, microorganism) and/or may be produced by biotechnological methods and/or other technologies.

The term “antibody” refers to a functional component of serum and is often referred to either as a collection of molecules (antibodies or immunoglobulins) or as one molecule (the antibody molecule or immunoglobulin molecule). An antibody is capable of binding to or reacting with a specific antigenic determinant (the antigen or the antigenic epitope), which in turn may lead to induction of immunological effector mechanisms. An individual antibody is usually regarded as monospecific, and a composition of antibodies may be monoclonal (i.e., consisting of identical antibody molecules) or polyclonal (i.e., consisting of two or more different antibodies reacting with the same or different epitopes on the same antigen or even on distinct, different antigens). Each antibody has a unique structure that enables it to bind specifically to its corresponding antigen, and all natural antibodies have the same overall basic structure of two identical light chains and two identical heavy chains. Antibodies are also known collectively as immunoglobulins. An antibody may be of human or non-human (for example, rodent such as murine, dog, camel, etc) origin (e.g., may have a sequence originally developed in a human or non-human cell or organism), or may be or comprise a chimeric, humanized, reshaped, or reformatted antibody based, e.g., on a such a human or non-human antibody (or, in some embodiments, on an antigen-binding portion thereof).

In some embodiments, as will be clear from context, the term “antibody” as used herein encompasses formats that include epitope-binding sequences of an antibody, which such formats include, for example chimeric and/or single chain antibodies (e.g., a nanobody or Fcab), as well as binding fragments of antibodies, such as Fab, Fv fragments or single chain Fv (scFv) fragments, as well as multimeric forms such as dimeric IgA molecules or pentavalent IgM molecules. Also included are bispecific antibodies, bispecific T cell engagers (BiTEs), immune mobilixing monoclonal T cell receptors against cancer (ImmTACs), dual-affinity re-targeting (DART); alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies.

The term “therapeutic agent” refers to an agent having one or more therapeutic properties that produce a desired, usually beneficial, effect. For example, a therapeutic agent may treat, ameliorate, and/or prevent disease. In some embodiments, a therapeutic agent may be or comprise a biologic, a small molecule, or a combination thereof.

The term “chemotherapeutic agent” refers to a therapeutic agent known to be of use in chemotherapy for cancer.

The term “cytokine” refers to a category of small proteins (˜5-25 kDa) important in cell signaling. Cytokines are peptides and have been shown to be involved in autocrine, paracrine, and endocrine signaling as immunomodulating agents. Cytokines include chemokines, interferons, interleukins, lymphokines, and tumour necrosis factors, but generally not hormones or growth factors. Class II cytokines include IL-10, IL-19, IL-20, IL-22, IL-24 (Mda-7), IL-26, type-I interferons (IFN-alpha, -beta, -epsilon, -kappa, -omega), type-II interferons (IFN-gamma), and type-III interferons (IFN-lambda, IL-28A, IL-28B, and IL-29).

The term “variant” encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Non-naturally-occurring variants include polypeptides and nucleic acids that comprise a change in amino acid or nucleotide sequence, respectively, where the change in sequence is artificially introduced (e.g., muteins); for example, the change is generated in the laboratory by human intervention. Thus, herein a “mutein” refers broadly to mutated recombinant proteins that usually carry single or multiple amino acid substitutions and are frequently derived from cloned genes that have been subjected to site-directed or random mutagenesis, or from completely synthetic genes.

### BRIEF DESCRIPTION OF THE DRAWINGS

FIGS. 1A-1B: Cytokine screen with anti CD47 antibody. FIGS. 1A and 1B are volcano plots summarizing the results of a cytokine screen in two different cancer cell types. Monocytes were isolated from peripheral blood mononuclear cells and differentiated into macrophages with M-CSF. Primary human macrophages were co-cultured in 384-well plates with GFP+ PC9 cancer cells (FIG. 1A; a human EGFR mutant non-small lung cancer cell line), or DLD-1 cancer cells (FIG. 1B; a human microsatellite unstable colon cancer cell line). Macrophages and cells were co-cultured in the absence or presence of an anti-CD47 antibody (B6H12, final concentration 10 ug/ml), with 114 cytokines at a concentration of 1 ug/ml as well as PBS control. Each cytokine condition was plated in triplicate. Cells were co-cultured for up to 7 days and GFP+ area was quantified by automated microscopy and image analysis (Incucyte®). Cell numbers per well: Human macrophages: 10,000 (1 donor, in triplicate); PC9: 2500; DLD-1:7500. FIG. 1A helps identify enhancers of CD47 blockade (e.g., IL-10). FIG. 1B identifies IFNW1 as an enhancer of macrophage cancer cytotoxicity independent of additional stimuli.

FIGS. 2A-2B: Validation of the combination of anti-CD47 antibody with IL-10. FIGS. 2A and 2B are plots summarizing the results of treating cancer cells with the combination of anti-CD47 antibody and IL-10 (FIG. 2A) or IL-10 alone (FIG. 2B). Primary human macrophages were co-cultured in 384-well plates with GFP+ PC9 cancer cells (a human EGFR mutant non-small lung cancer cell line). Macrophages and cells were co-cultured in the presence (FIG. 2A) or absence (FIG. 2B) of an anti-CD47 antibody (B6H12, final concentration 10 ug/ml), with IL-10 (final concentration 1 ug/ml) or PBS control. Cells were co-cultured for up to 7 days and GFP+ area was quantified by automated microscopy and image analysis (Incucyte®). Cell numbers per well: Human macrophages: 10,000 (3 donors, each in triplicate, two separate experiments); PC9: 2500.

FIGS. 3A-3B: EC₅₀ of the anti-CD47 antibody/IL-10 combination is in the nanomolar range. FIG. 3A and FIG. 3B show plots of titration curves of the anti-CD47 antibody/IL-10 combination in cancer cells. Primary human macrophages were co-cultured in 384-well plates with GFP+ PC9 cancer cells (a human EGFR mutant non-small lung cancer cell line). Macrophages and cells were co-cultured in the presence of an anti-CD47 antibody (B6H12, final concentration 10 ug/ml), with a range of IL-10 concentrations (1000 ng/ml-0.03 ng/ml). Cells were co-cultured for 4 days and GFP+ area was quantified by automated microscopy and image analysis (Incucyte®). EC₅₀ was calculated separately for each human macrophage donor. FIG. 3A and FIG. 3B each represents a separate human macrophage donor, 3 replicates per donor per concentration (EC₅₀=2.21 ng/ml and 3.55 ng/mL). Cell numbers per well: Human macrophages: 10,000; PC9: 2500.

FIGS. 4A-4B: IL-10 enhances macrophage-dependent 3LL inhibition in vitro. FIG. 4A and FIG. 4B show plots of the treatment of cancer cells with the combination of anti-CD47 antibody and IL-10, the combination of anti-CD47 antibody and interferon gamma, or control in mouse cancer cell models. Leukocytes were isolated from bone marrow and differentiated into macrophages with murine M-CSF. Murine C57BL/6 and NOD.Cg-Prkdc^{scid} Il2rg^{tm1Wjl}/SzJ (NSG) macrophages were co-cultured in 384-well plates with GFP+ 3LL ΔNRAS (murine lung cancer cell line) cancer cells. Macrophages and cells were co-cultured in the presence of an anti-CD47 antibody (MIAP410, final concentration 10 ug/ml) and either murine IL-10 (bottom curve in 4A and 4B), murine interferon gamma (middle curve in 4A and 4B) or control (top curve in 4A and 4B). Cells were co-cultured for up to 7 days and GFP+ area was quantified by automated microscopy and image analysis (Incucyte®). Each condition was plated in triplicate. Cell numbers per well: Murine macrophages: 10,000; 3LL: 1000.

FIGS. 5A-5B: Murine in vitro dose dependent responses to IL-10/anti-CD47 antibody. FIG. 5A and FIG. 5B show dose response curves of the treatment of cancer cells with the combination of anti-CD47 antibody and IL-10 in mouse cancer cell models. C57BL/6 or NSG macrophages were co-cultured in 384-well plates with GFP+ MC38 or 3LL cancer cells. Macrophages and cells were co-cultured in the presence of an anti-CD47 antibody (MIAP410, final concentration 10 ug/ml), with a range of IL-10 concentrations (1000 ng/ml-0.03 ng/ml). Cells were co-cultured for 4 days and GFP+ area was quantified by automated microscopy and image analysis (Incucyte®). EC₅₀ was calculated separately for C57BL/6 (0.022 ug/mL) and NSG (0.0339 ug/mL) macrophages. Each condition was done in triplicate. Cell numbers per well: Macrophages: 10,000; 3LL: 1000; MC38: 1000.

FIG. 6: Several members of the IL-10 cytokine family promote macrophage activation and cancer cell destruction in the presence of CD47 blockade. Primary human macrophages were co-cultured in 384-well plates with GFP+ PC9 cancer cells (a human EGFR mutant non-small lung cancer cell line). Macrophages and cells were co-cultured in the presence of an anti-CD47 antibody (B6H12, final concentration 10 ug/ml), and IL-10 family members (IL-10, IL-19, IL-20, IL-22, IL-24, IL-26, IL-28A, IL-28B, IL-29; final concentration 1 ug/ml) or PBS control. Cells were co-cultured for up to 7 days and GFP+ area was quantified by automated microscopy and image analysis (Incucyte®). Cell numbers per well: Human macrophages: 10,000 (2 donors, in triplicate); PC9: 2500.

FIG. 7: IL-10 combination with other antibodies/immune checkpoints enhances macrophage activity against lung cancer cells. Primary human macrophages were co-cultured in 384-well plates with GFP+ PC9 cancer cells (a human EGFR mutant non-small lung cancer cell line). Macrophages and cells were co-cultured in the presence of macrophage activating antibodies (final concentration 10 ug/ml) or control, with IL-10 (final concentration 100 ng/ml) or PBS control. Cells were co-cultured for up to 7 days and GFP+ area was quantified by automated microscopy and image analysis (Incucyte®). Cell numbers per well: Human macrophages: 10,000 (2 donors, each in triplicate); PC9: 2500. Macrophage activating antibodies: CD-40 agonist: clone G28.5; CD73 blockade: clone AD2; EGFR antibody: anti-hEGFR-hIgG1; PD-L1 antibody (silent Fc): Anti-hPD-L1-hIgG1 (N298A); PD-L2 antibody: clone MIH18.

FIG. 8: IL-10 combination with other antibodies/immune checkpoints may enhance macrophage activity against colon cancer cells. Primary human macrophages were co-cultured in 384-well plates with GFP+ DLD-1 cancer cells. Macrophages and cells were co-cultured in the presence of macrophage activating antibodies (final concentration 10 ug/ml) or control, with IL-10 (final concentration 100 ng/ml) or PBS control. Cells were co-cultured for up to 7 days and GFP+ area was quantified by automated microscopy and image analysis (Incucyte®). Cell numbers per well: Human macrophages: 10,000 (2 donors, each in triplicate); DLD-1:7500. Macrophage activating antibodies: CD-40 agonist: clone G28.5; CD73 blockade: clone AD2; EGFR antibody: anti-hEGFR-hIgG1; PD-L1 antibody (silent Fc): Anti-hPD-L1-hIgG1 (N298A); PD-L2 antibody: clone MIH18.

FIGS. 9A-9B: IL-10 combined with an opsonizing antibody enhances macrophage activity against lung cancer cells (PC9) and colon cancer cells (DLD-1). FIG. 9A. Primary human macrophages were co-cultured in 384-well plates with GFP+ PC9 cancer cells (a human EGFR mutant non-small lung cancer cell line). Macrophages and cells were co-cultured in the presence of macrophage activating antibodies (final concentration 10 ug/ml) or control, with IL-10 (final concentration 100 ng/ml) or PBS control. Cells were co-cultured for up to 7 days and GFP+ area was quantified by automated microscopy and image analysis (Incucyte®). Cell numbers per well: Human macrophages: 10,000 (2 donors, each in triplicate); PC9: 2500. Anti-EPCAM antibody (done with 3 donors, each in triplicate, clone 9C4). FIG. 9B. Primary human macrophages were co-cultured in 384-well plates with GFP+ DLD-1 cancer cells. Macrophages and cells were co-cultured in the presence of macrophage activating antibodies (final concentration 10 ug/ml) or control, with IL-10 (final concentration 100 ng/ml) or PBS control. Cells were co-cultured for up to 7 days and GFP+ area was quantified by automated microscopy and image analysis (Incucyte®). Cell numbers per well: Human macrophages: 10,000 (2 donors, each in triplicate); DLD-1:7500. Anti-EPCAM antibody (done with 3 donors, each in triplicate, clone 9C4)

FIG. 10: IL-10 priming of macrophages enhances anti-CD47 antibody induced phagocytosis of multiple cell lines. Primary human macrophages were primed with IL-10 (100 ng/ul) or PBS control for 48 hours. Cancer cells were stained with CFSE and macrophages were stained with an anti-CD45 APC antibody. Primed and unprimed macrophages (50K/well) were incubated with GFP+ cancers cells (200K/well) in the presence or absence of CD47 antibody (B6H12, final concentration 10 ug/ml) for 2 hours at 37° C. Phagocytosis was measured by flow cytometry as the percentage of macrophages containing engulfed CFSE+ cells. 8 human macrophage donors were tested with PC9 (lung cancer), H3122 (lung cancer), DLD-1 (colon cancer), and COLO205 (colon cancer). 2 human macrophage donors were tested with H358 (non-small cell lung cancer).

FIG. 11: Interferon alpha (IFN-α), interferon gamma (IFN-γ), and interferon omega (IFN-ω) activate macrophages to inhibit cancer cells. Primary human macrophages were co-cultured in 384-well plates with GFP+ DLD-1 cancer cells. Macrophages and cells were co-cultured in the presence of interferon alpha 2A (IFNA2A), interferon alpha 2B (IFNA2B), interferon omega (IFNW1) and interferon gamma (IFNG), all at a final concentration of 1000 ng/ml, or PBS control. GFP+ area was quantified at 7 days by automated microscopy and image analysis (Incucyte®). Cell numbers per well: Human macrophages: 10,000 (4 donors, each in triplicate); DLD-1:7500.

FIG. 12: IFN-ω can activate macrophages against cell lines with mesenchymal properties. Primary human macrophages were co-cultured in 384-well plates with GFP+ HMLER cancer cells with knockout of the EED or KMT2D genes (described in Zhang, et al. Nature Cell Biology, 2022, 24, 554-564, courtesy of the Weinberg Lab). Macrophages and cells were co-cultured in the presence of interferon omega (IFNW1) at a final concentration 1000 ng/ml, or PBS control. GFP+ area was quantified at 7 days by automated microscopy and image analysis (Incucyte®). Cell numbers per well: Human macrophages: 10,000 (3 donors, each in triplicate); HMLER: 7500.

FIG. 13: Human IFN-ω can activate murine macrophages against murine cancer cells. Murine C57BL/6 and NOD.Cg-Prkdc^{scid} Il2rg^{tm1Wjl}/SzJ (NSG) macrophages were co-cultured in 384-well plates with GFP+ MC38 cancer cells. Macrophages and cells were co-cultured in the presence of interferon omega (IFNW1), interferon gamma (IFNG), or PBS control. Cells were co-cultured for up to 7 days and GFP+ area was quantified by automated microscopy and image analysis (Incucyte®). Each condition was plated in triplicate. Cell numbers per well: Murine macrophages: 10,000; MC38: 1000.

FIG. 14: IL-10 enhances response to anti-CD47 therapy in a xenograft model of PC9 cancer cells engrafted into NSG (NOD scid gamma) mice. Human lung cancer PC9 cells were transduced with a lentiviral vector expressing murine IL-10 (IL10_OE) or control (Ctrl). 1 million PC9 IL10_OE or Ctrl in Matrigel were engrafted subcutaneously in NSG mice. Tumors were allowed to grow for 7 days and then mice were randomized to intraperitoneal treatment with vehicle control or B6H12 at a dose of 200 ug three times a week beginning on day 8. Mean tumor volume±SEM over time (in days) is shown. Bottom curve in graph is IL-10+anti-CD47 (lowest tumor volume at 44 days). Data represents a total of n=15 mice per group from 2 independent experiments. **, *** and **** indicate p≤0.01, ≤0.001 and ≤0.0001 by one way ANOVA with post-hoc Holm-Sidak correction at 44 days.

FIG. 15: IL-10 enhances response to anti-CD47 therapy in a syngeneic model of 3LL ΔNRAS cancer cells engrafted into NSG mice. Murine lung cancer 3LL ΔNRAS cells were transduced with a lentiviral vector expressing murine IL-10 (IL10_OE) or control (Ctrl). Five million 3LL ΔNRAS IL10_OE or Ctrl were engrafted subcutaneously in C57BL/6 mice. Tumors were allowed to grow for 6 days and then mice were randomized to intraperitoneal treatment with vehicle control or MIAP410. A priming dose of 50 ug of MIAP410 was given on day 7, followed by 200 ug beginning 2 days later for 2 additional doses. Mean tumor volume±SEM over time (in days) is shown. Bottom curve in graph is IL-10+anti-CD47 (lowest tumor volume at 14 days). Data represents a total of n=5 mice per group from one experiment.

FIGS. 16A-16B: IL-10 overexpression results in rejection of MC38 and KPCA cancer cells in immunocompetent mice. Murine ovarian cancer KPCA cells and murine colon cancer MC38 cells were transduced with a lentiviral vector expressing murine IL-10 (IL10_OE) or control (Ctrl). 1 million KPCA or MC38 IL10_OE or Ctrl were engrafted subcutaneously in C57BL/6 mice. Tumors were allowed to grow for 6 days (KPCA, FIG. 16A) or 8 days (MC38, FIG. 16B) and then mice were randomized to intraperitoneal treatment with vehicle control or MIAP410. A priming dose of 50 ug of MIAP410 was given on day 7 (KPCA) or 9 (MC38), followed by 200 ug three times a week beginning 2 days later. Mean tumor volume±SEM over time (in days) is shown. Data represents a total of n=5 mice per group from 1 experiment. ** indicates p≤0.01 by one way ANOVA with post-hoc Holm-Sidak correction at 22 days.

### DETAILED DESCRIPTION OF CERTAIN EMBODIMENTS

Provided herein are combination therapies employing an immunotherapy that stimulates macrophage phagocytosis of cancer cells. For example, the CD47/SIRPα axis is an immune checkpoint that regulates macrophage anti-tumor function. CD47 is ubiquitously expressed in human cells and has been found to be overexpressed in many different tumor cells. Therapies that block CD47 on cancer cells show promise in clinical trials for treating solid tumor and hematologic malignancies. Described herein are combination therapies that take advantage of and enhance macrophage phagocytosis of cancer cells to treat cancer.

The present disclosure describes the identification of several cytokine modulators of macrophage-mediated cancer cytotoxicity. A screen of 114 cytokines in a co-culture assay of human macrophages with human cancer cell lines was conducted. For this screen, two different cell lines, PC9 (derived from EGFR mutated lung adenocarcinoma) and DLD-1 (derived from microsatellite unstable colon cancer) were employed. Macrophages were differentiated from peripheral blood monocytes. Each cytokine was also tested in the presence of an antibody targeting CD47, a known macrophage immune checkpoint. This approach led to the discovery of context-dependent enhancers and inhibitors of macrophage activity.

Specifically, interleukin 10 (IL-10) was identified as a potentiator of macrophage-mediated cancer cytotoxicity in the context of CD47 blockade. The present discloure demonstrates that other members of the IL-10 subfamily (e.g., IL-26, interferon lambda 3) can also potentiate CD47 blockade. Interferon omega (IFN-ω) was also identified as a potentiator of macrophage cytotoxicity. Initial in vitro results described herein translated well to mouse cancer models, including the mouse 3LL (Lewis lung carcinoma) and MC38 (colon adenocarcinoma) cell lines in co-culture assays with both NSG and C57BL/6 macrophages.

Moreover, identifying IL-10 as a potentiator of CD47 blockade antibody dependent cytotoxicity is a surprising and unexpected result since studies have shown that IL-10 is a macrophage inhibitor. Accordingly, in one embodiment, disclosed herein is a surprising and unexpected therapeutic strategy to enhance the efficacy of anti-CD47 therapies by combining them with cytokines. Thus, combining a macrophage-directed immunotherapy with a cytokine may improve treatment efficacy and confer survival benefit in patients with cancer.

### Methods of Treatment

One aspect of the present disclosure relates to methods of treating a proliferative disease in a subject in need thereof. In certain embodiments, the proliferative disease is cancer. The methods include administering a macrophage-directed immunotherapy and a cytokine. The methods also include administering a bifunctional compound comprising a macrophage-directed immunotherapy and a cytokine, or a modification, fragment, or variant thereof.

In another aspect, the present disclosure provides methods of treating a cancer in a subject in need thereof, the methods comprising administering to the subject an effective amount (e.g., therapeutically effective amount) of (1) a macrophage-directed immunotherapy and a cytokine described herein, (2) a bifunctional compound described herein, or (3) a pharmaceutical composition described herein. In certain embodiments, the macrophage-directed immunotherapy and cytokine are synergistic in treating the cancer, compared to the macrophage-directed immunotherapy and/or cytokine alone.

In another aspect, the present disclosure provides methods of preventing a cancer in a subject in need thereof, the methods comprising administering to the subject an effective amount (e.g., prophylactically effective amount) of (1) a macrophage-directed immunotherapy and a cytokine described herein, (2) a bifunctional compound described herein, or (3) a pharmaceutical composition described herein. In certain embodiments, the macrophage-directed immunotherapy and cytokine are synergistic in preventing the cancer, compared to the macrophage-directed immunotherapy and/or cytokine alone.

In another aspect, the present disclosure provides methods of reducing, delaying, and/or preventing in a subject in need thereof the resistance of a cancer to a macrophage-directed immunotherapy and/or cytokine, the methods comprising administering to the subject an effective amount of (1) a macrophage-directed immunotherapy and a cytokine described herein, (2) a bifunctional compound described herein, or (3) a pharmaceutical composition described herein. In certain embodiments, the macrophage-directed immunotherapy and cytokine are synergistic in reducing, delaying, and/or preventing the resistance of the cancer to the macrophage-directed immunotherapy and/or cytokine, compared to the macrophage-directed immunotherapy and/or cytokine alone.

In certain embodiments, the macrophage-directed immunotherapy and cytokine are administered to the subject at the same time. In certain embodiments, the macrophage-directed immunotherapy and cytokine are administered to the subject at different times.

In another aspect, the present disclosure provides methods of inhibiting the proliferation of a cell, the methods comprising contacting the cell with an effective amount of (1) a macrophage-directed immunotherapy and a cytokine described herein, (2) a bifunctional compound described herein, or (3) a pharmaceutical composition described herein. In certain embodiments, the macrophage-directed immunotherapy and cytokine are synergistic in inhibiting the proliferation of the cell, compared to the macrophage-directed immunotherapy and/or cytokine alone.

In another aspect, the present disclosure provides methods of reducing, delaying, and/or preventing the resistance of a cell to a macrophage-directed immunotherapy and/or cytokine, the methods comprising contacting the cell with an effective amount of (1) a macrophage-directed immunotherapy and a cytokine described herein, (2) a bifunctional compound described herein, or (3) a pharmaceutical composition described herein. In certain embodiments, the macrophage-directed immunotherapy and cytokine are synergistic in reducing, delaying, and/or preventing the resistance of the cell to the macrophage-directed immunotherapy and/or cytokine, compared to the macrophage-directed immunotherapy and/or cytokine alone.

In another aspect, the present disclosure provides the macrophage-directed immunotherapies and cytokines described herein for use in a method described herein (e.g., a method of treating cancer in a subject in need thereof, a method of preventing a cancer in a subject in need thereof, a method of reducing, delaying, and/or preventing in a subject in need thereof the resistance of a cancer to a macrophage-directed immunotherapy and/or cytokine, a method of inhibiting the proliferation of a cell, or a method of reducing, delaying, and/or preventing the resistance of a cell to a macrophage-directed immunotherapy and/or cytokine). In certain embodiments, the present disclosure provides the macrophage-directed immunotherapies and cytokines for use in treating cancer in a subject in need thereof. In certain embodiments, the present disclosure provides a combination of the macrophage-directed immunotherapies and cytokines for use in treating a cancer in a subject in need thereof.

In still another aspect, the present disclosure provides the pharmaceutical compositions described herein for use in a method described herein (e.g., a method of treating cancer in a subject in need thereof, a method of preventing a cancer in a subject in need thereof, a method of reducing, delaying, and/or preventing in a subject in need thereof the resistance of a cancer to a macrophage-directed immunotherapy and/or cytokine, a method of inhibiting the proliferation of a cell, or a method of reducing, delaying, and/or preventing the resistance of a cell to a macrophage-directed immunotherapy and/or cytokine). In certain embodiments, the present disclosure provides the pharmaceutical compositions for use in treating cancer in a subject in need thereof.

In certain embodiments, the methods described herein result in an increase in phagocytosis of cancer cells compared to treatment with the cytokine alone. In certain embodiments, the methods described herein result in an increase in phagocytosis of cancer cells compared to treatment with the macrophage-directed immunotherapy alone. In certain embodiments, the methods described herein result in a synergistic increase in phagocytosis of cancer cells compared to treatment with the macrophage-directed immunotherapy and/or the cytokine alone. In certain embodiments, the increase in phagocytosis of cancer cells is observed in a biological sample from a subject. In certain embodiments, the increase in phagocytosis of cancer cells is observed in an in vitro experiment. In certain embodiments, the cancer cells are lung cancer cells. In certain embodiments, the cancer cells are non-small cell lung cancer cells. In certain embodiments, the cancer cells are small cell lung cancer cells. In certain embodiments, the cancer cells are ovarian cancer cells. In certain embodiments, the cancer cells are colon cancer cells. In certain embodiments, the cancer cells are colorectal cancer cells.

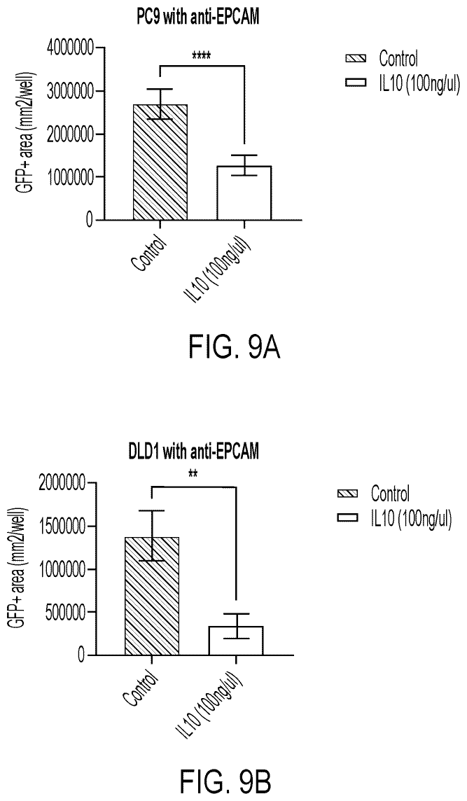

In certain embodiments, the methods comprise ex vivo priming of macrophages with the cytokine (e.g., IL-10) and subsequently administering the primed macrophages. In such embodiments, macrophages are primed by the cytokine for 1, 2, 3, 4, 8, 12, 18, 24, 48, 72, or 96 hours. In certain embodiments, the methods comprise administering the primed macrophages prior to administering the macrophage-directed immunotherapy. In certain embodiments, the methods comprise administering the macrophage-directed immunotherapy prior to administering the primed macrophages. In certain embodiments, the methods comprise administering the primed macrophages at the same time as administering the macrophage-directed immunotherapy. In certain embodiments, the methods comprise administering additional cytokine after administering the primed macrophages. In certain embodiments, the methods do not comprise administering additional cytokine after administering the primed macrophages. In certain embodiments, macrophages are isolated from a subject, which are primed by the cytokine as described herein. In certain embodiments, macrophages are isolated from a subject, the macrophages are primed by the cytokine as described herein, and the primed macrophages are administered to the same subject. In certain embodiments, the macrophages are isolated for priming from a different source, i.e., not the subject being treated (e.g., a compatible donor). In any of the foregoing embodiments, the macrophages for priming are differentiated from monocytes. In certain embodiments, the macrophages are genetically engineered cells. In certain embodiments, the macrophages (or monocytes that could be differentiated into macrophages ex vivo) are isolated from a subject, exposed to IL-10 ex vivo, and then administered to the subject (who may have been and/or is subsequently treated with a macrophage-directed immunotherapy). The subject may or may not be further treated with IL-10. The macrophages (or monocytes) for priming may be isolated from a compatible donor. For example, see FIG. 10.

In certain embodiments, the treatment results in an increase of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% in phagocytosis of cancer cells compared to treatment with the macrophage-directed immunotherapy and/or the cytokine alone. In certain embodiments, the treatment results in at least a 2-fold, at least a 3-fold, at least a 4-fold, at least a 5-fold, at least a 6-fold, at least a 7-fold, at least a 8-fold, at least a 9-fold, at least a 10-fold, at least a 20-fold, at least a 30-fold, at least a 40-fold, at least a 50-fold, at least a 60-fold, at least a 70-fold, at least a 80-fold, at least a 90-fold, at least a 100-fold, at least a 1000-fold, at least a 10000-fold, or at least a 100000-fold increase in phagocytosis of cancer cells compared to treatment with the macrophage-directed immunotherapy and/or the cytokine alone.

In certain embodiments, the cancer cells are lung cancer cells. In certain embodiments, the cancer cells are non-small cell lung cancer cells. In certain embodiments, the cancer cells are small cell lung cancer cells. In certain embodiments, the cancer cells are ovarian cancer cells. In certain embodiments, the cancer cells are colon cancer cells. In certain embodiments, the cancer cells are colorectal cancer cells.

In certain embodiments, the macrophage-directed immunotherapies and cytokines, or pharmaceutical compositions thereof, can be administered in combination with an anti-cancer therapy including, but not limited to, surgery, radiation therapy, transplantation (e.g., stem cell transplantation, bone marrow transplantation), and chemotherapy. In certain embodiments the macrophage-directed immunotherapies and cytokines, or pharmaceutical compositions thereof, can be administered in combination with chemotherapy (i.e., one or more chemotherapeutic agents).

The methods described herein may be used to treat any cancer.

In certain embodiments, the cancer is a cancer that is commonly treated with chemotherapy. In certain embodiments, the cancer is a cancer that is commonly treated with immunotherapy. In some embodiments, the cancer is or comprises a solid tumor or hematological malignancy. In some embodiments, the cancer is or comprises a solid tumor. In some embodiments, the cancer is or comprises a hematological malignancy. In some embodiments, the cancer is a leukemia; a lymphoma; myelodysplasia; multiple myeloma; lung cancer (e.g., bronchogenic carcinoma, small cell lung cancer (SCLC), non-small cell lung cancer (NSCLC), adenocarcinoma of the lung); kidney cancer; acoustic neuroma; adenocarcinoma; adrenal gland cancer; anal cancer; angiosarcoma; appendix cancer; benign monoclonal gammopathy; biliary cancer; bladder cancer; breast cancer; brain cancer; bronchus cancer; carcinoid tumor; cervical cancer; choriocarcinoma; chordoma; craniopharyngioma; colorectal cancer; connective tissue cancer; epithelial carcinoma; ependymoma; endotheliosarcoma; endometrial cancer; esophageal cancer; Ewing's sarcoma; ocular cancer; familiar hypereosinophilia; gall bladder cancer; gastric cancer; gastrointestinal stromal tumor (GIST); germ cell cancer; head and neck cancer; heavy chain disease; leiomyosarcoma (LMS); mastocytosis; muscle cancer; myelodysplastic syndrome (MDS); mesothelioma; myeloproliferative disorder (MPD); neuroblastoma; neurofibroma; neuroendocrine cancer; osteosarcoma; ovarian cancer; papillary adenocarcinoma; pancreatic cancer; penile cancer; pinealoma; primitive neuroectodermal tumor (PNT); plasma cell neoplasia; paraneoplastic syndromes; intraepithelial neoplasms; prostate cancer; rectal cancer; rhabdomyosarcoma; salivary gland cancer; skin cancer; small bowel cancer; soft tissue sarcoma; sebaceous gland carcinoma; small intestine cancer; sweat gland carcinoma; synovioma; testicular cancer; thyroid cancer; urethral cancer; vaginal cancer; or vulvar cancer.

In certain embodiments, the cancer is bladder cancer, cervical cancer, dermatofibrosarcoma protuberans, endocrine tumors, neuroendocrine tumors, neuroblastoma, lung cancer (e.g., non-small cell lung cancer), anaplastic large cell lymphoma, glioblastoma multiforme, bile duct cancer, stomach cancer, colon cancer, rectal cancer, melanoma, colorectal cancer, brain cancer, head and neck cancer, thyroid cancer, soft tissue cancer, colon cancer, kidney cancer (e.g., papillary renal carcinoma), liver cancer, gastric cancer, gastrointestinal stromal tumor, giant cell tumor, esophageal cancer, gastroesophageal cancer, breast cancer, ovarian cancer, prostate cancer, endometrial cancer, pancreatic cancer, leukemia (e.g., acute myeloid leukemia), lymphoma, multiple myeloma, colon adenocarcinoma, lung adenocarcinoma, cutaneous melanoma, gastrointestinal cancer, anal cancer, glioblastoma, epithelian tumors of the head and neck, laryngeal cancer, oral cancer, myelodysplastic disorders, myeloproliferative disorders, ovarian epithelial cancer, fallopian tube cancer, primary peritoneal cancer, plexiform neurofibroma, skin cancer, soft tissue sarcoma, solid tumors with an NTRK gene fusion, or systemic mastocytosis.

In certain embodiments, the cancer is neuroblastoma, lung cancer (e.g., non-small cell lung cancer), anaplastic large cell lymphoma, glioblastoma multiforme, bile duct cancer, stomach cancer, colon cancer, rectal cancer, melanoma, colorectal cancer, brain cancer, head and neck cancer, thyroid cancer, soft tissue cancer, colon cancer, kidney cancer (e.g., papillary renal carcinoma), liver cancer, gastric cancer, gastroesophageal cancer, breast cancer, ovarian cancer, prostate cancer, endometrial carcinoma, pancreatic cancer, leukemia (e.g., acute myeloid leukemia), colon adenocarcinoma, lung adenocarcinoma, cutaneous melanoma, gastrointestinal cancer, anal cancer, glioblastoma, epithelian tumors of the head and neck, laryngeal cancer, and oral cancer. In certain embodiments, the cancer is breast cancer. In certain embodiments, the cancer is colorectal cancer. In certain embodiments, the cancer is colon cancer. In certain embodiments, the cancer is ovarian cancer. In certain embodiments, the cancer is lung cancer. In some embodiments, the cancer is non-small cell lung cancer (NSCLC). In some embodiments, the cancer is small cell lung cancer.

In certain embodiments, the cancer is a cancer that is commonly treated with a targeted agent. In certain embodiments, the cancer is a cancer with a driver mutation that can be treated with a targeted agent directed at that driver mutation.

In certain embodiments, the cancer is associated with microsatellite instability such as colon cancer and endometrial cancer.

In certain embodiments, the cancer is associated with overexpressed and/or mutated EGFR, such as non-small cell lung cancer, adenocarcinoma of the lung, anal cancer, glioblastoma, and epithelian tumors of the head and neck.

### Macrophage-Directed Immunotherapy

As described herein, a macrophage-directed immunotherapy is an immunotherapy that activates macrophages and derives its therapeutic effect by stimulating macrophages. Such stimulation can mobilize macrophage and myeloid components to destroy a tumor and its stroma, including the tumor vasculature. Macrophages can be induced to secrete antitumor cytokines and/or to perform phagocytosis, including antibody-dependent cellular phagocytosis.

In certain embodiments, the macrophage-directed immunotherapy is an immunotherapeutic agent. In certain embodiments, the macrophage-directed immunotherapy is a macrophage immune checkpoint inhibitor. In certain embodiments, the immunotherapeutic agent is a macrophage immune checkpoint inhibitor.

As described herein, a macrophage immune checkpoint inhibitor functions to stimulate macrophage phagocytosis of cancer cells. For example, CD47 is associated with a macrophage immune checkpoint (CD47/SIRPα as described herein).

In certain embodiments, the macrophage-directed immunotherapy is a small molecule. In certain embodiments, the macrophage-directed immunotherapy is a biologic. In certain embodiments, the biologic is a protein. In certain embodiments, the biologic is an antibody or fragment thereof. In certain embodiments, the biologic is a nucleic acid that encodes a protein.

In certain embodiments, the macrophage-directed immunotherapy comprises modulation of CD47, SIRPα, EGFR, MHC I, CD24, CALR, CD40, PD-L1, APMAP, GPR84, VCAM1, CD11b, SIGLEC-10, PD-L2, PD-1, CD73, epCAM, Galectin-9, CD14, CD80, CD86, SIRPb, SIRPg, SLAMF7, MARCO, AXL, CLEVER-1, ILT4, TIM-3, TIM-4, LRP-1, calreticulin, TREM1, TREM2, GD2, FcgRI, FcgRIIa, FcgRIIb, FcgRIII, MUC1, CD44, CD63, CD36, CD84, CD164, CD82, CD18, SIGLEC-7, CD166, CD39, CD46, LILRA1, LILRA2(ILT1), LILRA3 (ILT6), LILRA4 (ILT7), LILRB1 (ILT2), LILRB2 (ILT4), LILRB3 (ILT5), LILRB4 (ILT3), LILRB5, CD85b (ILT8 or ILT9), CD85m (ILT10), CD85f (ILT11), CD276, CD88, CD99, PILRa, Siglec-9, CD206, CD163, CD84 (SLAMF5), C3aR, or CLEC12A. In certain embodiments, the macrophage-directed immunotherapy comprises modulation of CD47, SIRPα, EGFR, CD40, PD-L1, PD-L2, CD73, or EpCAM.

In certain embodiments, the macrophage-directed immunotherapy is a CD47 inhibitor, a SIRPα inhibitor, an EGFR inhibitor, an MHC I inhibitor, a CD24 inhibitor, a CALR inhibitor, a CD40 agonist, a PD-L1 inhibitor, an APMAP inhibitor, a GPR84 inhibitor, a VCAM1 inhibitor, a CD11b inhibitor, a SIGLEC-10 inhibitor, a PD-L2 inhibitor, a PD-1 inhibitor, a CD73 inhibitor, an EpCAM inhibitor, a Galectin-9 inhibitor, a CD14 inhibitor, a CD80 inhibitor, a CD86 inhibitor, a SIRPb inhibitor, a SIRPg inhibitor, a SLAMF7 inhibitor, a MARCO inhibitor, an AXL inhibitor, a CLEVER-1 inhibitor, an ILT4 inhibitor, a TIM-3 inhibitor, a TIM-4 inhibitor, an LRP-1 inhibitor, a calreticulin inhibitor, a TREM1 inhibitor, a TREM2 inhibitor, a GD2 inhibitor, an FcgRI inhibitor, an FcgRIIa inhibitor, an FcgRIIb inhibitor, an FcgRIII inhibitor, a MUC1 inhibitor, a CD44 inhibitor, a CD63 inhibitor, a CD36 inhibitor, a CD84 inhibitor, a CD164 inhibitor, a CD82 inhibitor, a CD18 inhibitor, a SIGLEC-7 inhibitor, a CD166 inhibitor, a CD39 inhibitor, a CD46 inhibitor, an LILRA1 inhibitor, an LILRA2 inhibitor, an LILRA3 inhibitor, an LILRA4 inhibitor, an LILRB1 inhibitor, an LILRB2 inhibitor, an LILRB3 inhibitor, an LILRB4 inhibitor, an LILRB5 inhibitor, a CD85b inhibitor, a CD85m inhibitor, a CD85f inhibitor, a CD276 inhibitor, a CD88 inhibitor, a CD99 inhibitor, a PILRa inhibitor, a Siglec-9 inhibitor, a CD206 inhibitor, a CD163 inhibitor, a CD84 inhibitor, a C3aR inhibitor, or a CLEC12A inhibitor.

In certain embodiments, the macrophage-directed immunotherapy is a CD47 inhibitor, a SIRPα inhibitor, an EGFR inhibitor, a CD40 agonist, a PD-L1 inhibitor, a PD-L2 inhibitor, a CD73 inhibitor, or an EpCAM inhibitor. In certain embodiments, the macrophage-directed immunotherapy is a CD47 inhibitor or a SIRPα inhibitor. In certain embodiments, the macrophage-directed immunotherapy is a CD47 inhibitor and a SIRPα inhibitor. In certain embodiments, the macrophage-directed immunotherapy is a CD47 inhibitor. In certain embodiments, the macrophage-directed immunotherapy is a SIRPα inhibitor.

In certain embodiments, the macrophage-directed immunotherapy is an anti-CD47 antibody, an anti-SIRPα antibody, a SIRPα-Fc fusion protein, an anti-EGFR antibody, an anti-MHC I antibody, an anti-CD24 antibody, an anti-CALR antibody, an anti-CD40 antibody, an anti-PD-L1 antibody, an anti-APMAP antibody, an anti-GPR84 antibody, an anti-VCAM1 antibody, an anti-CD11b antibody, an anti-SIGLEC-10 antibody, an anti-PD-L2 antibody, an anti-PD-1 antibody, an anti-CD73 antibody, an anti-EpCAM antibody, an anti-Galectin-9 antibody, an anti-CD14 antibody, an anti-CD80 antibody, an anti-CD86 antibody, an anti-SIRPb antibody, an anti-SIRPg antibody, an anti-SLAMF7 antibody, an anti-MARCO antibody, an anti-AXL antibody, an anti-CLEVER-1 antibody, an anti-ILT4 antibody, an anti-TIM-3 antibody, an anti-TIM-4 antibody, an anti-LRP-1 antibody, an anti-calreticulin antibody, an anti-TREM1 antibody, an anti-TREM2 antibody, an anti-GD2 antibody, an anti-FcgRI antibody, an anti-FcgRIIa antibody, an anti-FcgRIIb antibody, an anti-FcgRIII antibody, an anti-MUC1 antibody, an anti-CD44 antibody, an anti-CD63 antibody, an anti-CD36 antibody, an anti-CD84 antibody, an anti-CD164 antibody, an anti-CD82 antibody, an anti-CD18 antibody, an anti-SIGLEC-7 antibody, an anti-CD166 antibody, an anti-CD39 antibody, an anti-CD46 antibody, an anti-LILRA1 antibody, an anti-LILRA2 antibody, an anti-LILRA3 antibody, an anti-LILRA4 antibody, an anti-LILRB1 antibody, an anti-LILRB2 antibody, an anti-LILRB3 antibody, an anti-LILRB4 antibody, an anti-LILRB5 antibody, an anti-CD85b antibody, an anti-CD85m antibody, an anti-CD85f antibody, an anti-CD276 antibody, an anti-CD88 antibody, an anti-CD99 antibody, an anti-PILRa antibody, an anti-Siglec-9 antibody, an anti-CD206 antibody, an anti-CD163 antibody, an anti-CD84 antibody, an anti-C3aR antibody, or an anti-CLEC12A antibody.

In certain embodiments, the macrophage-directed immunotherapy is an anti-CD47 antibody, an anti-SIRPα antibody, a SIRPα-Fc fusion protein, an anti-EGFR antibody, an anti-CD40 antibody, an anti-PD-L1 antibody, an anti-PD-L2 antibody, an anti-CD73 antibody, or an anti-EpCAM antibody. In certain embodiments, the macrophage-directed immunotherapy is an anti-CD47 antibody, a SIRPα-Fc fusion protein, or an anti-SIRPα antibody. In certain embodiments, the macrophage-directed immunotherapy is an anti-CD47 antibody or an anti-SIRPα antibody. In certain embodiments, the macrophage-directed immunotherapy is a SIRPα-Fc fusion protein. In certain embodiments, the macrophage-directed immunotherapy is an anti-SIRPα antibody. In certain embodiments, the macrophage-directed immunotherapy is an anti-CD47 antibody.

In certain embodiments, the macrophage-directed immunotherapy is magrolimab, TTI-621, TTI-622, AO-176, HX-009, AK117, AK112, CC90002, STI-6643, PF-07257876, IMC-002, CPO107, SRF231, IBI188, IBI322, IMM2902, BAT7104, TG-1801, SL-172154, BI 765063, TQB2928, or GS-0189.

In certain embodiments, the macrophage-directed immunotherapy is magrolimab. In certain embodiments, the macrophage-directed immunotherapy is TTI-621. In certain embodiments, the macrophage-directed immunotherapy is TTI-622. In certain embodiments, the macrophage-directed immunotherapy is AO-176. In certain embodiments, the macrophage-directed immunotherapy is HX-009. In certain embodiments, the macrophage-directed immunotherapy is AK117. In certain embodiments, the macrophage-directed immunotherapy is AK112. In certain embodiments, the macrophage-directed immunotherapy is CC90002. In certain embodiments, the macrophage-directed immunotherapy is STI-6643. In certain embodiments, the macrophage-directed immunotherapy is PF-07257876. In certain embodiments, the macrophage-directed immunotherapy is TQB2928. In certain embodiments, the macrophage-directed immunotherapy is IMC-002. In certain embodiments, the macrophage-directed immunotherapy is CPO107. In certain embodiments, the macrophage-directed immunotherapy is SRF231. In certain embodiments, the macrophage-directed immunotherapy is IBI188. In certain embodiments, the macrophage-directed immunotherapy is IBI322. In certain embodiments, the macrophage-directed immunotherapy is IMM2902. In certain embodiments, the macrophage-directed immunotherapy is BAT7104. In certain embodiments, the macrophage-directed immunotherapy is TG-1801. In certain embodiments, the macrophage-directed immunotherapy is SL-172154. In certain embodiments, the macrophage-directed immunotherapy is BI 765063. In certain embodiments, the macrophage-directed immunotherapy is GS-0189.

In certain embodiments, the macrophage-directed immunotherapy comprises any antibody with an Fc that interacts with the Fc receptor on macrophages and stimulates the macrophages via Fc receptor engagement. In certain embodiments, the antibody is rituximab, trastuzumab, cetuximab, or panitumumab.

### Cytokine

The methods disclosed herein comprise administering a cytokine. In certain embodiments, the cytokine includes modifications, fragments, and variants thereof.

In certain embodiments, the cytokine is an anti-inflammatory cytokine, or a modification, fragment, or variant thereof. In certain embodiments, the cytokine is a class II cytokine, or a modification, fragment, or variant thereof. Class II cytokines include interleukin-10 (IL-10), interleukin-19 (IL-19), interleukin-20 (IL-20), interleukin-22 (IL-22), interleukin-24 (IL-24), interleukin-26 (IL-26), type-I interferons (e.g., interferon alpha (IFN-α), interferon beta (IFN-β), interferon epsilon (IFN-ε), interferon kappa (IFN-κ), interferon omega (IFN-ω)), type-II interferons (e.g., interferon gamma (IFN-γ)), and type-III interferons (e.g., interleukin-28A (IL-28A), interleukin-28B (IL-28B), interleukin-29 (IL-29)).

In certain embodiments, the cytokine is interleukin-10 (IL-10), interleukin-19 (IL-19), interleukin-20 (IL-20), interleukin-22 (IL-22), interleukin-24 (IL-24), interleukin-26 (IL-26), interleukin-28A (IL-28A), interleukin-28B (IL-28B), interleukin-29 (IL-29), a type-I interferon, a type-II interferon, or a modification, fragment, or variant thereof.

In certain embodiments, the cytokine is interleukin-10 (IL-10), interleukin-19 (IL-19), interleukin-20 (IL-20), interleukin-22 (IL-22), interleukin-24 (IL-24), interleukin-26 (IL-26), interleukin-28A (IL-28A), interleukin-28B (IL-28B), or interleukin-29 (IL-29), or a modification, fragment, or variant thereof.

In certain embodiments, the cytokine is interleukin-10 (IL-10), or a modification, fragment, or variant thereof. In certain embodiments, the amino acid sequence listing of IL-10 is MSPGQGTQSE NSCTHFPGNL PNMLRDLRDA FSRVKTFFQM KDQLDNLLLK ESLLEDFKGY LGCQALSEMI QFYLEEVMPQ AENQDPDIKA HVNSLGENLK TLRLRLRRCH RFLPCENKSK AVEQVKNAFN KLQEKGIYKA MSEFDIFINY IEAYMTMKIR N. In certain embodiments, the cytokine is interleukin-19 (IL-19), or a modification, fragment, or variant thereof. In certain embodiments, the cytokine is interleukin-20 (IL-20), or a modification, fragment, or variant thereof. In certain embodiments, the cytokine is interleukin-22 (IL-22), or a modification, fragment, or variant thereof. In certain embodiments, the cytokine is interleukin-24 (IL-24), or a modification, fragment, or variant thereof. In certain embodiments, the cytokine is interleukin-26 (IL-26), or a modification, fragment, or variant thereof. In certain embodiments, the cytokine is interleukin-28A (IL-28A), or a modification, fragment, or variant thereof. In certain embodiments, the cytokine is interleukin-28B (IL-28B), or a modification, fragment, or variant thereof. In certain embodiments, the cytokine is interleukin-29 (IL-29), or a modification, fragment, or variant thereof.

In certain embodiments, the cytokine is a type-I interferon or a type-II interferon, or a modification, fragment, or variant thereof. In certain embodiments, the cytokine is a type-I interferon, or a modification, fragment, or variant thereof. In certain embodiments, the cytokine is IFN-α, IFN-β, IFN-ε, IFN-κ, or IFN-ω or a modification, fragment, or variant thereof. In certain embodiments, the cytokine is IFN-α or IFN-ω, or a modification, fragment, or variant thereof. In certain embodiments, the cytokine is IFN-α2A, IFN-α2B, or IFN-ω, or a modification, fragment, or variant thereof. In certain embodiments, the cytokine is IFN-α, or a modification, fragment, or variant thereof. In certain embodiments, the cytokine is IFN-α2A, or a modification, fragment, or variant thereof. In certain embodiments, the cytokine is IFN-α2B, or a modification, fragment, or variant thereof. In certain embodiments, the cytokine is IFN-ω, or a modification, fragment, or variant thereof. In certain embodiments, the cytokine is a type-II interferon, or a modification, fragment, or variant thereof. In certain embodiments, the cytokine is IFN-γ, or a modification, fragment, or variant thereof. In certain embodiments, the cytokine is IFN-γ, IFN-α, or IFN-ω, or a modification, fragment, or variant thereof. In certain embodiments, the cytokine is IFN-γ, IFN-α2A, IFN-α2B, or IFN-ω, or a modification, fragment, or variant thereof.

Certain embodiments of the present disclosure contemplate modification of a cytokine (e.g., IL-10) in order to enhance one or more properties (e.g., pharmacokinetic parameters, efficacy). In certain embodiments, a cytokine (e.g., IL-10) is modified chemically, i.e., a chemical moiety is attached to the cytokine by formation of new chemical bonds. In certain embodiments, a cytokine (e.g., IL-10) is modified by, for example, pegylation, glycosylation, albumin (e.g., human serum albumin (HSA)) conjugation, and/or hesylation. In further embodiments, modification of a cytokine (e.g., IL-10) does not result in a therapeutically relevant, detrimental effect on immunogenicity, and in still further embodiments a modified cytokine (e.g., IL-10) is less immunogenic than an unmodified cytokine (e.g., IL-10).

In certain embodiments, the cytokine includes human and non-human forms, including homologs, variants (including muteins), and fragments thereof, as well as polypeptides having, for example, a leader sequence (e.g., the signal peptide), and modified versions of the foregoing (i.e., a modification). In certain embodiments, the cytokine is pegylated. In certain embodiments, the cytokine is pegylated IL-10. In certain embodiments, the cytokine is pegilodecakin.

In certain embodiments, the cytokine is a variant of any cytokine described herein. In certain embodiments, the cytokine is a variant of IL-10. In certain embodiments, the cytokine variant has at least 80%, 85%, 90%, 95%, 97%, 98%, 99%, or 99.5% sequence identity to the amino acid sequence of any cytokine described herein. In certain embodiments, the cytokine variant has at least 80%, 85%, 90%, 95%, 97%, 98%, 99%, or 99.5% sequence identity to the amino acid sequence of IL-10. In certain embodiments, the cytokine variant has at least 80%, 85%, 90%, 95%, 97%, 98%, 99%, or 99.5% sequence identity to the amino acid sequence of human IL-10.

### Certain Embodiments

In certain embodiments, the method comprises administering IL-10 and a macrophage-directed immunotherapy. In certain embodiments, the method comprises administering IL-10 and an immunotherapeutic agent. In certain embodiments, the method comprises administering IL-10 and a macrophage immune checkpoint inhibitor. In certain embodiments, the method comprises administering IL-10 and a CD47 inhibitor, a SIRPα inhibitor, an EGFR inhibitor, a CD40 agonist, a PD-L1 inhibitor, a PD-L2 inhibitor, a CD73 inhibitor, or an EpCAM inhibitor. In certain embodiments, the method comprises administering IL-10 and a CD47 inhibitor or a SIRPα inhibitor. In certain embodiments, the method comprises administering IL-10 and a CD47 inhibitor. In certain embodiments, the method comprises administering IL-10 and an anti-CD47 antibody, an anti-SIRPα antibody, a SIRPα-Fc fusion protein, an anti-EGFR antibody, an anti-CD40 antibody, an anti-PD-L1 antibody, an anti-PD-L2 antibody, an anti-CD73 antibody, or an anti-EpCAM antibody. In certain embodiments, the method comprises administering IL-10 and an anti-CD47 antibody. In certain embodiments, the method comprises administering IL-10 and magrolimab, TTI-621, TTI-622, AO-176, HX-009, AK117, AK112, CC90002, STI-6643, PF-07257876, IMC-002, CPO107, SRF231, TQB2928, IBI188, IBI322, IMM2902, BAT7104, TG-1801, SL-172154, BI 765063, or GS-0189.

### Bifunctional Compounds

Another aspect of the present disclosure relates to bifunctional compounds, and pharmaceutically acceptable salts thereof, that comprise a macrophage-directed immunotherapy and a cytokine, or a modification, fragment, or variant thereof. In certain embodiments, the macrophage-directed immunotherapy is any macrophage-directed immunotherapy described herein; and the cytokine is any cytokine described herein.

In certain embodiments, the bifunctional compound is a fusion protein. In certain embodiments, at least one domain of the fusion protein is derived from the macrophage-directed immunotherapy and at least one domain of the fusion protein is derived from the cytokine.

In certain embodiments, the macrophage-directed immunotherapy and cytokine are attached through a linker. In certain embodiments, the linker is a covalent linker, i.e., the macrophage-directed immunotherapy and cytokine are covalently linked. In certain embodiments, the linker is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; and each R^{A} is independently hydrogen, a protecting group, or substituted or unsubstituted alkyl.

In certain embodiments, the macrophage-directed immunotherapy is an immunotherapeutic agent. In certain embodiments, the macrophage-directed immunotherapy is a macrophage immune checkpoint inhibitor. In certain embodiments, the immunotherapeutic agent is a macrophage immune checkpoint inhibitor.

In certain embodiments, the macrophage-directed immunotherapy is a small molecule. In certain embodiments, the macrophage-directed immunotherapy is a biologic. In certain embodiments, the biologic is a protein. In certain embodiments, the biologic is an antibody or fragment thereof. In certain embodiments, the biologic is a nucleic acid that encodes a protein.

In certain embodiments, the macrophage-directed immunotherapy comprises modulation of CD47, SIRPα, EGFR, MHC I, CD24, CALR, CD40, PD-L1, APMAP, GPR84, VCAM1, CD11b, SIGLEC-10, PD-L2, PD-1, CD73, epCAM, Galectin-9, CD14, CD80, CD86, SIRPb, SIRPg, SLAMF7, MARCO, AXL, CLEVER-1, ILT4, TIM-3, TIM-4, LRP-1, calreticulin, TREM1, TREM2, GD2, FcgRI, FcgRIIa, FcgRIIb, FcgRIII, MUC1, CD44, CD63, CD36, CD84, CD164, CD82, CD18, SIGLEC-7, CD166, CD39, CD46, LILRA1, LILRA2(ILT1), LILRA3 (ILT6), LILRA4 (ILT7), LILRB1 (ILT2), LILRB2 (ILT4), LILRB3 (ILT5), LILRB4 (ILT3), LILRB5, CD85b (ILT8 or ILT9), CD85m (ILT10), CD85f (ILT11), CD276, CD88, CD99, PILRa, Siglec-9, CD206, CD163, CD84 (SLAMF5), C₃aR, or CLEC12A. In certain embodiments, the macrophage-directed immunotherapy comprises modulation of CD47, SIRPα, EGFR, CD40, PD-L1, PD-L2, CD73, or EpCAM.

In certain embodiments, the macrophage-directed immunotherapy is a CD47 inhibitor, a SIRPα inhibitor, an EGFR inhibitor, an MHC I inhibitor, a CD24 inhibitor, a CALR inhibitor, a CD40 agonist, a PD-L1 inhibitor, an APMAP inhibitor, a GPR84 inhibitor, a VCAM1 inhibitor, a CD11b inhibitor, a SIGLEC-10 inhibitor, a PD-L2 inhibitor, a PD-1 inhibitor, a CD73 inhibitor, an EpCAM inhibitor, a Galectin-9 inhibitor, a CD14 inhibitor, a CD80 inhibitor, a CD86 inhibitor, a SIRPb inhibitor, a SIRPg inhibitor, a SLAMF7 inhibitor, a MARCO inhibitor, an AXL inhibitor, a CLEVER-1 inhibitor, an ILT4 inhibitor, a TIM-3 inhibitor, a TIM-4 inhibitor, an LRP-1 inhibitor, a calreticulin inhibitor, a TREM1 inhibitor, a TREM2 inhibitor, a GD2 inhibitor, an FcgRI inhibitor, an FcgRIIa inhibitor, an FcgRIIb inhibitor, an FcgRIII inhibitor, a MUC1 inhibitor, a CD44 inhibitor, a CD63 inhibitor, a CD36 inhibitor, a CD84 inhibitor, a CD164 inhibitor, a CD82 inhibitor, a CD18 inhibitor, a SIGLEC-7 inhibitor, a CD166 inhibitor, a CD39 inhibitor, a CD46 inhibitor, an LILRA1 inhibitor, an LILRA2 inhibitor, an LILRA3 inhibitor, an LILRA4 inhibitor, an LILRB1 inhibitor, an LILRB2 inhibitor, an LILRB3 inhibitor, an LILRB4 inhibitor, an LILRB5 inhibitor, a CD85b inhibitor, a CD85m inhibitor, a CD85f inhibitor, a CD276 inhibitor, a CD88 inhibitor, a CD99 inhibitor, a PILRa inhibitor, a Siglec-9 inhibitor, a CD206 inhibitor, a CD163 inhibitor, a CD84 inhibitor, a C₃aR inhibitor, or a CLEC12A inhibitor.

In certain embodiments, the macrophage-directed immunotherapy is a CD47 inhibitor, a SIRPα inhibitor, an EGFR inhibitor, a CD40 agonist, a PD-L1 inhibitor, a PD-L2 inhibitor, a CD73 inhibitor, or an EpCAM inhibitor. In certain embodiments, the macrophage-directed immunotherapy is a CD47 inhibitor or a SIRPα inhibitor. In certain embodiments, the macrophage-directed immunotherapy is a CD47 inhibitor and a SIRPα inhibitor. In certain embodiments, the macrophage-directed immunotherapy is a CD47 inhibitor. In certain embodiments, the macrophage-directed immunotherapy is a SIRPα inhibitor.

In certain embodiments, the macrophage-directed immunotherapy is an anti-CD47 antibody, an anti-SIRPα antibody, a SIRPα-Fc fusion protein, an anti-EGFR antibody, an anti-MHC I antibody, an anti-CD24 antibody, an anti-CALR antibody, an anti-CD40 antibody, an anti-PD-L1 antibody, an anti-APMAP antibody, an anti-GPR84 antibody, an anti-VCAM1 antibody, an anti-CD11b antibody, an anti-SIGLEC-10 antibody, an anti-PD-L2 antibody, an anti-PD-1 antibody, an anti-CD73 antibody, an anti-EpCAM antibody, an anti-Galectin-9 antibody, an anti-CD14 antibody, an anti-CD80 antibody, an anti-CD86 antibody, an anti-SIRPb antibody, an anti-SIRPg antibody, an anti-SLAMF7 antibody, an anti-MARCO antibody, an anti-AXL antibody, an anti-CLEVER-1 antibody, an anti-ILT4 antibody, an anti-TIM-3 antibody, an anti-TIM-4 antibody, an anti-LRP-1 antibody, an anti-calreticulin antibody, an anti-TREM1 antibody, an anti-TREM2 antibody, an anti-GD2 antibody, an anti-FcgRI antibody, an anti-FcgRIIa antibody, an anti-FcgRIIb antibody, an anti-FcgRIII antibody, an anti-MUC1 antibody, an anti-CD44 antibody, an anti-CD63 antibody, an anti-CD36 antibody, an anti-CD84 antibody, an anti-CD164 antibody, an anti-CD82 antibody, an anti-CD18 antibody, an anti-SIGLEC-7 antibody, an anti-CD166 antibody, an anti-CD39 antibody, an anti-CD46 antibody, an anti-LILRA1 antibody, an anti-LILRA2 antibody, an anti-LILRA3 antibody, an anti-LILRA4 antibody, an anti-LILRB1 antibody, an anti-LILRB2 antibody, an anti-LILRB3 antibody, an anti-LILRB4 antibody, an anti-LILRB5 antibody, an anti-CD85b antibody, an anti-CD85m antibody, an anti-CD85f antibody, an anti-CD276 antibody, an anti-CD88 antibody, an anti-CD99 antibody, an anti-PILRa antibody, an anti-Siglec-9 antibody, an anti-CD206 antibody, an anti-CD163 antibody, an anti-CD84 antibody, an anti-C3aR antibody, or an anti-CLEC12A antibody.

In certain embodiments, the macrophage-directed immunotherapy is an anti-CD47 antibody, an anti-SIRPα antibody, a SIRPα-Fc fusion protein, an anti-EGFR antibody, an anti-CD40 antibody, an anti-PD-L1 antibody, an anti-PD-L2 antibody, an anti-CD73 antibody, or an anti-EpCAM antibody. In certain embodiments, the macrophage-directed immunotherapy is an anti-CD47 antibody, a SIRPα-Fc fusion protein, or an anti-SIRPα antibody. In certain embodiments, the macrophage-directed immunotherapy is an anti-CD47 antibody or an anti-SIRPα antibody. In certain embodiments, the macrophage-directed immunotherapy is a SIRPα-Fc fusion protein. In certain embodiments, the macrophage-directed immunotherapy is an anti-SIRPα antibody. In certain embodiments, the macrophage-directed immunotherapy is an anti-CD47 antibody.

In certain embodiments, the macrophage-directed immunotherapy is magrolimab, TTI-621, TTI-622, AO-176, HX-009, AK117, AK112, CC90002, STI-6643, PF-07257876, IMC-002, CPO107, SRF231, IBI188, IBI322, IMM2902, BAT7104, TG-1801, SL-172154, BI 765063, TQB2928, or GS-0189.

In certain embodiments, the macrophage-directed immunotherapy is magrolimab. In certain embodiments, the macrophage-directed immunotherapy is TTI-621. In certain embodiments, the macrophage-directed immunotherapy is TTI-622. In certain embodiments, the macrophage-directed immunotherapy is AO-176. In certain embodiments, the macrophage-directed immunotherapy is HX-009. In certain embodiments, the macrophage-directed immunotherapy is AK117. In certain embodiments, the macrophage-directed immunotherapy is AK112. In certain embodiments, the macrophage-directed immunotherapy is CC90002. In certain embodiments, the macrophage-directed immunotherapy is STI-6643. In certain embodiments, the macrophage-directed immunotherapy is PF-07257876. In certain embodiments, the macrophage-directed immunotherapy is TQB2928. In certain embodiments, the macrophage-directed immunotherapy is IMC-002. In certain embodiments, the macrophage-directed immunotherapy is CPO107. In certain embodiments, the macrophage-directed immunotherapy is SRF231. In certain embodiments, the macrophage-directed immunotherapy is IBI188. In certain embodiments, the macrophage-directed immunotherapy is IBI322. In certain embodiments, the macrophage-directed immunotherapy is IMM2902. In certain embodiments, the macrophage-directed immunotherapy is BAT7104. In certain embodiments, the macrophage-directed immunotherapy is TG-1801. In certain embodiments, the macrophage-directed immunotherapy is SL-172154. In certain embodiments, the macrophage-directed immunotherapy is BI 765063. In certain embodiments, the macrophage-directed immunotherapy is GS-0189.

In certain embodiments, the macrophage-directed immunotherapy comprises any antibody with an Fc that interacts with the Fc receptor on macrophages and stimulates the macrophages via Fc receptor engagement. In certain embodiments, the antibody is rituximab, trastuzumab, cetuximab, or panitumumab.

In certain embodiments, the cytokine includes modifications, fragments, and variants thereof. In certain embodiments, the cytokine is an anti-inflammatory cytokine, or a modification, fragment, or variant thereof. In certain embodiments, the cytokine is a class II cytokine, or a modification, fragment, or variant thereof. Class II cytokines include interleukin-10 (IL-10), interleukin-19 (IL-19), interleukin-20 (IL-20), interleukin-22 (IL-22), interleukin-24 (IL-24), interleukin-26 (IL-26), type-I interferons (e.g., interferon alpha (IFN-α), interferon beta (IFN-β), interferon epsilon (IFN-ε), interferon kappa (IFN-κ), interferon omega (IFN-ω)), type-II interferons (e.g., interferon gamma (IFN-γ)), and type-III interferons (e.g., interleukin-28A (IL-28A), interleukin-28B (IL-28B), interleukin-29 (IL-29)).

In certain embodiments, the cytokine is interleukin-10 (IL-10), interleukin-19 (IL-19), interleukin-20 (IL-20), interleukin-22 (IL-22), interleukin-24 (IL-24), interleukin-26 (IL-26), interleukin-28A (IL-28A), interleukin-28B (IL-28B), interleukin-29 (IL-29), a type-I interferon, a type-II interferon, or a modification, fragment, or variant thereof.

In certain embodiments, the cytokine is interleukin-10 (IL-10), interleukin-19 (IL-19), interleukin-20 (IL-20), interleukin-22 (IL-22), interleukin-24 (IL-24), interleukin-26 (IL-26), interleukin-28A (IL-28A), interleukin-28B (IL-28B), or interleukin-29 (IL-29), or a modification, fragment, or variant thereof.

In certain embodiments, the cytokine is interleukin-10 (IL-10), or a modification, fragment, or variant thereof. In certain embodiments, the cytokine is interleukin-19 (IL-19), or a modification, fragment, or variant thereof. In certain embodiments, the cytokine is interleukin-20 (IL-20), or a modification, fragment, or variant thereof. In certain embodiments, the cytokine is interleukin-22 (IL-22), or a modification, fragment, or variant thereof. In certain embodiments, the cytokine is interleukin-24 (IL-24), or a modification, fragment, or variant thereof. In certain embodiments, the cytokine is interleukin-26 (IL-26), or a modification, fragment, or variant thereof. In certain embodiments, the cytokine is interleukin-28A (IL-28A), or a modification, fragment, or variant thereof. In certain embodiments, the cytokine is interleukin-28B (IL-28B), or a modification, fragment, or variant thereof. In certain embodiments, the cytokine is interleukin-29 (IL-29), or a modification, fragment, or variant thereof.

In certain embodiments, the cytokine is a type-I interferon or a type-II interferon, or a modification, fragment, or variant thereof. In certain embodiments, the cytokine is a type-I interferon, or a modification, fragment, or variant thereof. In certain embodiments, the cytokine is IFN-α, IFN-β, IFN-ε, IFN-κ, or IFN-ω, or a modification, fragment, or variant thereof. In certain embodiments, the cytokine is IFN-α or IFN-ω, or a modification, fragment, or variant thereof. In certain embodiments, the cytokine is IFN-α2A, IFN-α2B, or IFN-ω, or a modification, fragment, or variant thereof. In certain embodiments, the cytokine is IFN-α, or a modification, fragment, or variant thereof. In certain embodiments, the cytokine is IFN-α2A, or a modification, fragment, or variant thereof. In certain embodiments, the cytokine is IFN-α2B, or a modification, fragment, or variant thereof. In certain embodiments, the cytokine is IFN-ω, or a modification, fragment, or variant thereof. In certain embodiments, the cytokine is a type-II interferon, or a modification, fragment, or variant thereof. In certain embodiments, the cytokine is IFN-γ, or a modification, fragment, or variant thereof. In certain embodiments, the cytokine is IFN-γ, IFN-α, or IFN-ω, or a modification, fragment, or variant thereof. In certain embodiments, the cytokine is IFN-γ, IFN-α2A, IFN-α2B, or IFN-ω, or a modification, fragment, or variant thereof.

Certain embodiments of the present disclosure contemplate modification of a cytokine (e.g., IL-10) in order to enhance one or more properties (e.g., pharmacokinetic parameters, efficacy). In certain embodiments, a cytokine (e.g., IL-10) is modified chemically, i.e., a chemical moiety is attached to the cytokine by formation of new chemical bonds. In certain embodiments, a cytokine (e.g., IL-10) is modified by, for example, pegylation, glycosylation, albumin (e.g., human serum albumin (HSA)) conjugation, and hesylation. In further embodiments, modification of a cytokine (e.g., IL-10) does not result in a therapeutically relevant, detrimental effect on immunogenicity, and in still further embodiments modification of a cytokine (e.g., IL-10) is less immunogenic than unmodified cytokine (e.g., IL-10).

In certain embodiments, the cytokine includes human and non-human forms, including homologs, variants (including muteins), and fragments thereof, as well as polypeptides having, for example, a leader sequence (e.g., the signal peptide), and modified versions of the foregoing. In certain embodiments, the cytokine is pegylated. In certain embodiments, the cytokine is pegylated IL-10. In certain embodiments, the cytokine is pegilodecakin.

In certain embodiments, the cytokine is a variant of any cytokine described herein. In certain embodiments, the cytokine is a variant of IL-10. In certain embodiments, the cytokine variant has at least 80%, 85%, 90%, 95%, 97%, 98%, 99%, or 99.5% sequence identity to the amino acid sequence of any cytokine described herein. In certain embodiments, the cytokine variant has at least 80%, 85%, 90%, 95%, 97%, 98%, 99%, or 99.5% sequence identity to the amino acid sequence of IL-10. In certain embodiments, the cytokine variant has at least 80%, 85%, 90%, 95%, 97%, 98%, 99%, or 99.5% sequence identity to the amino acid sequence of human IL-10.

In certain embodiments, the bifunctional compound is an anti-CD47 antibody and IL-10 attached through a linker. In certain embodiments, the bifunctional compound is an anti-CD47 antibody and a modified IL-10 attached through a linker. In certain embodiments, the bifunctional compound is an anti-CD47 antibody and a variant of IL-10 attached through a linker. In certain embodiments, the bifunctional compound is an anti-CD47 antibody and a fragment of IL-10 attached through a linker.

### Pharmaceutical Compositions, Kits, and Administration

Another aspect of the present disclosure relates to pharmaceutical compositions that comprise a macrophage-directed immunotherapy and a cytokine, and optionally a pharmaceutically acceptable excipient. In certain embodiments, the pharmaceutical composition comprises a bifunctional compound, or pharmaceutically acceptable salt thereof, described herein.

The pharmaceutical compositions described herein may be useful in treating and/or preventing cancer in a subject in need thereof, such as cancers that are resistant to or are at risk of becoming resistant to a cytokine and/or a macrophage-directed immunotherapy. The pharmaceutical compositions described herein may also be useful in reducing, delaying, and/or preventing in a subject in need thereof, the resistance of a cancer to treatment with a cytokine and/or a macrophage-directed immunotherapy. The pharmaceutical compositions described herein may further be useful in inhibiting the proliferation of a cell, and/or reducing, delaying, and/or preventing the resistance of a cell to a cytokine and/or a macrophage-directed immunotherapy. The pharmaceutical compositions described herein are expected to be synergistic in treating and/or preventing cancer in the subject; in reducing, delaying, and/or preventing the resistance of cancer in the subject to a cytokine and/or a macrophage-directed immunotherapy; in inhibiting the proliferation of the cell, and/or reducing, delaying, and/or preventing the resistance of the cell to a cytokine and/or a macrophage-directed immunotherapy, compared to the cytokine and/or the macrophage-directed immunotherapy alone.

A pharmaceutical composition described herein comprises a macrophage-directed immunotherapy. In certain embodiments, the macrophage-directed immunotherapy is any macrophage-directed immunotherapy as described herein. In certain embodiments, the macrophage-directed immunotherapy is an immunotherapeutic agent. In certain embodiments, the macrophage-directed immunotherapy is a macrophage immune checkpoint inhibitor.

In certain embodiments, the macrophage-directed immunotherapy comprises modulation of CD47, SIRPα, EGFR, MHC I, CD24, CALR, CD40, PD-L1, APMAP, GPR84, VCAM1, CD11b, SIGLEC-10, PD-L2, PD-1, CD73, epCAM, Galectin-9, CD14, CD80, CD86, SIRPb, SIRPg, SLAMF7, MARCO, AXL, CLEVER-1, ILT4, TIM-3, TIM-4, LRP-1, calreticulin, TREM1, TREM2, GD2, FcgRI, FcgRIIa, FcgRIIb, FcgRIII, MUC1, CD44, CD63, CD36, CD84, CD164, CD82, CD18, SIGLEC-7, CD166, CD39, CD46, LILRA1, LILRA2(ILT1), LILRA3 (ILT6), LILRA4 (ILT7), LILRB1 (ILT2), LILRB2 (ILT4), LILRB3 (ILT5), LILRB4 (ILT3), LILRB5, CD85b (ILT8 or ILT9), CD85m (ILT10), CD85f (ILT11), CD276, CD88, CD99, PILRa, Siglec-9, CD206, CD163, CD84 (SLAMF5), C3aR, or CLEC12A. In certain embodiments, the macrophage-directed immunotherapy comprises modulation of CD47, SIRPα, EGFR, CD40, PD-L1, PD-L2, CD73, or EpCAM.

In certain embodiments, the macrophage-directed immunotherapy is a CD47 inhibitor, a SIRPα inhibitor, an EGFR inhibitor, an MHC I inhibitor, a CD24 inhibitor, a CALR inhibitor, a CD40 agonist, a PD-L1 inhibitor, an APMAP inhibitor, a GPR84 inhibitor, a VCAM1 inhibitor, a CD11b inhibitor, a SIGLEC-10 inhibitor, a PD-L2 inhibitor, a PD-1 inhibitor, a CD73 inhibitor, an EpCAM inhibitor, a Galectin-9 inhibitor, a CD14 inhibitor, a CD80 inhibitor, a CD86 inhibitor, a SIRPb inhibitor, a SIRPg inhibitor, a SLAMF7 inhibitor, a MARCO inhibitor, an AXL inhibitor, a CLEVER-1 inhibitor, an ILT4 inhibitor, a TIM-3 inhibitor, a TIM-4 inhibitor, an LRP-1 inhibitor, a calreticulin inhibitor, a TREM1 inhibitor, a TREM2 inhibitor, a GD2 inhibitor, an FcgRI inhibitor, an FcgRIIa inhibitor, an FcgRIIb inhibitor, an FcgRIII inhibitor, a MUC1 inhibitor, a CD44 inhibitor, a CD63 inhibitor, a CD36 inhibitor, a CD84 inhibitor, a CD164 inhibitor, a CD82 inhibitor, a CD18 inhibitor, a SIGLEC-7 inhibitor, a CD166 inhibitor, a CD39 inhibitor, a CD46 inhibitor, an LILRA1 inhibitor, an LILRA2 inhibitor, an LILRA3 inhibitor, an LILRA4 inhibitor, an LILRB1 inhibitor, an LILRB2 inhibitor, an LILRB3 inhibitor, an LILRB4 inhibitor, an LILRB5 inhibitor, a CD85b inhibitor, a CD85m inhibitor, a CD85f inhibitor, a CD276 inhibitor, a CD88 inhibitor, a CD99 inhibitor, a PILRa inhibitor, a Siglec-9 inhibitor, a CD206 inhibitor, a CD163 inhibitor, a CD84 inhibitor, a C3aR inhibitor, or a CLEC12A inhibitor. In certain embodiments, the macrophage-directed immunotherapy is a CD47 inhibitor, a SIRPα inhibitor, an EGFR inhibitor, a CD40 agonist, a PD-L1 inhibitor, a PD-L2 inhibitor, a CD73 inhibitor, or an EpCAM inhibitor.

In certain embodiments, the macrophage-directed immunotherapy is a CD47 inhibitor or a SIRPα inhibitor. In certain embodiments, the macrophage-directed immunotherapy is a CD47 inhibitor and a SIRPα inhibitor. In certain embodiments, the macrophage-directed immunotherapy is a CD47 inhibitor. In certain embodiments, the macrophage-directed immunotherapy is a SIRPα inhibitor.

In certain embodiments, the macrophage-directed immunotherapy is a biologic. In certain embodiments, the macrophage-directed immunotherapy is an antibody or antibody fragment.

In certain embodiments, the macrophage-directed immunotherapy is an anti-CD47 antibody, an anti-SIRPα antibody, a SIRPα-Fc fusion protein, an anti-EGFR antibody, an anti-MHC I antibody, an anti-CD24 antibody, an anti-CALR antibody, an anti-CD40 antibody, an anti-PD-L1 antibody, an anti-APMAP antibody, an anti-GPR84 antibody, an anti-VCAM1 antibody, an anti-CD11b antibody, an anti-SIGLEC-10 antibody, an anti-PD-L2 antibody, an anti-PD-1 antibody, an anti-CD73 antibody, an anti-EpCAM antibody, an anti-Galectin-9 antibody, an anti-CD14 antibody, an anti-CD80 antibody, an anti-CD86 antibody, an anti-SIRPb antibody, an anti-SIRPg antibody, an anti-SLAMF7 antibody, an anti-MARCO antibody, an anti-AXL antibody, an anti-CLEVER-1 antibody, an anti-ILT4 antibody, an anti-TIM-3 antibody, an anti-TIM-4 antibody, an anti-LRP-1 antibody, an anti-calreticulin antibody, an anti-TREM1 antibody, an anti-TREM2 antibody, an anti-GD2 antibody, an anti-FcgRI antibody, an anti-FcgRIIa antibody, an anti-FcgRIIb antibody, an anti-FcgRIII antibody, an anti-MUC1 antibody, an anti-CD44 antibody, an anti-CD63 antibody, an anti-CD36 antibody, an anti-CD84 antibody, an anti-CD164 antibody, an anti-CD82 antibody, an anti-CD18 antibody, an anti-SIGLEC-7 antibody, an anti-CD166 antibody, an anti-CD39 antibody, an anti-CD46 antibody, an anti-LILRA1 antibody, an anti-LILRA2 antibody, an anti-LILRA3 antibody, an anti-LILRA4 antibody, an anti-LILRB1 antibody, an anti-LILRB2 antibody, an anti-LILRB3 antibody, an anti-LILRB4 antibody, an anti-LILRB5 antibody, an anti-CD85b antibody, an anti-CD85m antibody, an anti-CD85f antibody, an anti-CD276 antibody, an anti-CD88 antibody, an anti-CD99 antibody, an anti-PILRa antibody, an anti-Siglec-9 antibody, an anti-CD206 antibody, an anti-CD163 antibody, an anti-CD84 antibody, an anti-C3aR antibody, or an anti-CLEC12A antibody. In certain embodiments, the macrophage-directed immunotherapy is an anti-CD47 antibody, an anti-SIRPα antibody, a SIRPα-Fc fusion protein, an anti-EGFR antibody, an anti-CD40 antibody, an anti-PD-L1 antibody, an anti-PD-L2 antibody, an anti-CD73 antibody, or an anti-EpCAM antibody. In certain embodiments, the macrophage-directed immunotherapy is an anti-CD47 antibody, a SIRPα-Fc fusion protein, or an anti-SIRPα antibody. In certain embodiments, the macrophage-directed immunotherapy is an anti-CD47 antibody or an anti-SIRPα antibody. In certain embodiments, the macrophage-directed immunotherapy is a SIRPα-Fc fusion protein. In certain embodiments, the macrophage-directed immunotherapy is an anti-SIRPα antibody. In certain embodiments, the macrophage-directed immunotherapy is an anti-CD47 antibody.

In certain embodiments, the macrophage-directed immunotherapy is magrolimab, TTI-621, TTI-622, AO-176, HX-009, AK117, AK112, CC90002, STI-6643, PF-07257876, IMC-002, CPO107, SRF231, IBI188, IBI322, IMM2902, BAT7104, TG-1801, SL-172154, BI 765063, TQB2928, or GS-0189.

In certain embodiments, the macrophage-directed immunotherapy comprises any antibody with an Fc that interacts with the Fc receptor on macrophages and stimulates the macrophages via Fc receptor engagement. In certain embodiments, the antibody is rituximab, trastuzumab, cetuximab, or panitumumab.

In certain embodiments, the macrophage-directed immunotherapy is magrolimab. In certain embodiments, the macrophage-directed immunotherapy is TTI-621. In certain embodiments, the macrophage-directed immunotherapy is TTI-622. In certain embodiments, the macrophage-directed immunotherapy is AO-176. In certain embodiments, the macrophage-directed immunotherapy is HX-009. In certain embodiments, the macrophage-directed immunotherapy is AK117. In certain embodiments, the macrophage-directed immunotherapy is AK112. In certain embodiments, the macrophage-directed immunotherapy is CC90002. In certain embodiments, the macrophage-directed immunotherapy is STI-6643. In certain embodiments, the macrophage-directed immunotherapy is PF-07257876. In certain embodiments, the macrophage-directed immunotherapy is TQB2928. In certain embodiments, the macrophage-directed immunotherapy is IMC-002. In certain embodiments, the macrophage-directed immunotherapy is CPO107. In certain embodiments, the macrophage-directed immunotherapy is SRF231. In certain embodiments, the macrophage-directed immunotherapy is IBI188. In certain embodiments, the macrophage-directed immunotherapy is IBI322. In certain embodiments, the macrophage-directed immunotherapy is IMM2902. In certain embodiments, the macrophage-directed immunotherapy is BAT7104. In certain embodiments, the macrophage-directed immunotherapy is TG-1801. In certain embodiments, the macrophage-directed immunotherapy is SL-172154. In certain embodiments, the macrophage-directed immunotherapy is BI 765063. In certain embodiments, the macrophage-directed immunotherapy is GS-0189.

A pharmaceutical composition described herein further comprises a cytokine. In certain embodiments, the cytokine includes modifications, fragments, and variants thereof.

In certain embodiments, the cytokine is an anti-inflammatory cytokine, or a modification, fragment, or variant thereof. In certain embodiments, the cytokine is a class II cytokine, or a modification, fragment, or variant thereof. Class II cytokines include interleukin-10 (IL-10), interleukin-19 (IL-19), interleukin-20 (IL-20), interleukin-22 (IL-22), interleukin-24 (IL-24), interleukin-26 (IL-26), type-I interferons (e.g., interferon alpha (IFN-α), interferon beta (IFN-β), interferon epsilon (IFN-ε), interferon kappa (IFN-κ), interferon omega (IFN-ω)), type-II interferons (e.g., interferon gamma (IFN-γ)), and type-III interferons (e.g., interleukin-28A (IL-28A), interleukin-28B (IL-28B), interleukin-29 (IL-29)).

In certain embodiments, the cytokine is interleukin-10 (IL-10), interleukin-19 (IL-19), interleukin-20 (IL-20), interleukin-22 (IL-22), interleukin-24 (IL-24), interleukin-26 (IL-26), interleukin-28A (IL-28A), interleukin-28B (IL-28B), interleukin-29 (IL-29), a type-I interferon, a type-II interferon, or a modification, fragment, or variant thereof.

In certain embodiments, the cytokine is interleukin-10 (IL-10), interleukin-19 (IL-19), interleukin-20 (IL-20), interleukin-22 (IL-22), interleukin-24 (IL-24), interleukin-26 (IL-26), interleukin-28A (IL-28A), interleukin-28B (IL-28B), or interleukin-29 (IL-29), or a modification, fragment, or variant thereof.

In certain embodiments, the cytokine is interleukin-10 (IL-10), or a modification, fragment, or variant thereof. In certain embodiments, the cytokine is interleukin-19 (IL-19), or a modification, fragment, or variant thereof. In certain embodiments, the cytokine is interleukin-20 (IL-20), or a modification, fragment, or variant thereof. In certain embodiments, the cytokine is interleukin-22 (IL-22), or a modification, fragment, or variant thereof. In certain embodiments, the cytokine is interleukin-24 (IL-24), or a modification, fragment, or variant thereof. In certain embodiments, the cytokine is interleukin-26 (IL-26), or a modification, fragment, or variant thereof. In certain embodiments, the cytokine is interleukin-28A (IL-28A), or a modification, fragment, or variant thereof. In certain embodiments, the cytokine is interleukin-28B (IL-28B), or a modification, fragment, or variant thereof. In certain embodiments, the cytokine is interleukin-29 (IL-29), or a modification, fragment, or variant thereof.

In certain embodiments, the cytokine is a type-I interferon or a type-II interferon, or a modification, fragment, or variant thereof. In certain embodiments, the cytokine is a type-I interferon, or a modification, fragment, or variant thereof. In certain embodiments, the cytokine is IFN-α, IFN-β, IFN-ε, IFN-κ, or IFN-ω or a modification, fragment, or variant thereof. In certain embodiments, the cytokine is IFN-α or IFN-ω, or a modification, fragment, or variant thereof. In certain embodiments, the cytokine is IFN-α2A, IFN-α2B, or IFN-ω, or a modification, fragment, or variant thereof. In certain embodiments, the cytokine is IFN-α, or a modification, fragment, or variant thereof. In certain embodiments, the cytokine is IFN-α2A, or a modification, fragment, or variant thereof. In certain embodiments, the cytokine is IFN-α2B, or a modification, fragment, or variant thereof. In certain embodiments, the cytokine is IFN-ω, or a modification, fragment, or variant thereof. In certain embodiments, the cytokine is a type-II interferon, or a modification, fragment, or variant thereof. In certain embodiments, the cytokine is IFN-γ, or a modification, fragment, or variant thereof. In certain embodiments, the cytokine is IFN-γ, IFN-α, or IFN-ω, or a modification, fragment, or variant thereof. In certain embodiments, the cytokine is IFN-γ, IFN-α2A, IFN-α2B, or IFN-ω, or a modification, fragment, or variant thereof.

Certain embodiments of the present disclosure contemplate modification of a cytokine (e.g., IL-10) in order to enhance one or more properties (e.g., pharmacokinetic parameters, efficacy). In certain embodiments, a cytokine (e.g., IL-10) is modified chemically, i.e., a chemical moiety is attached to the cytokine by formation of new chemical bonds. In certain embodiments, a cytokine (e.g., IL-10) is modified by, for example, pegylation, glycosylation, albumin (e.g., human serum albumin (HSA)) conjugation, and hesylation. In further embodiments, modification of a cytokine (e.g., IL-10) does not result in a therapeutically relevant, detrimental effect on immunogenicity, and in still further embodiments modification of a cytokine (e.g., IL-10) is less immunogenic than unmodified cytokine (e.g., IL-10).

In certain embodiments, the cytokine includes human and non-human forms, including homologs, variants (including muteins), and fragments thereof, as well as polypeptides having, for example, a leader sequence (e.g., the signal peptide), and modified versions of the foregoing. In certain embodiments, the cytokine is pegylated. In certain embodiments, the cytokine is pegylated IL-10. In certain embodiments, the cytokine is pegilodecakin.

In certain embodiments, the cytokine is a variant of any cytokine described herein. In certain embodiments, the cytokine is a variant of IL-10. In certain embodiments, the cytokine variant has at least 80%, 85%, 90%, 95%, 97%, 98%, 99%, or 99.5% sequence identity to the amino acid sequence of any cytokine described herein. In certain embodiments, the cytokine variant has at least 80%, 85%, 90%, 95%, 97%, 98%, 99%, or 99.5% sequence identity to the amino acid sequence of IL-10. In certain embodiments, the cytokine variant has at least 80%, 85%, 90%, 95%, 97%, 98%, 99%, or 99.5% sequence identity to the amino acid sequence of human IL-10.

A pharmaceutical composition described herein may further comprise one or more chemotherapeutic agents. In certain embodiments, the chemotherapeutic agent is any chemotherapeutic agent as described herein.

In certain embodiments, the pharmaceutical composition comprises IL-10 and a macrophage-directed immunotherapy. In certain embodiments, the pharmaceutical composition comprises IL-10 and an immunotherapeutic agent. In certain embodiments, the pharmaceutical composition comprises IL-10 and a macrophage immune checkpoint inhibitor. In certain embodiments, the pharmaceutical composition comprises IL-10 and a CD47 inhibitor, a SIRPα inhibitor, an EGFR inhibitor, a CD40 agonist, a PD-L1 inhibitor, a PD-L2 inhibitor, a CD73 inhibitor, or an EpCAM inhibitor. In certain embodiments, the pharmaceutical composition comprises IL-10 and a CD47 inhibitor or a SIRPα inhibitor. In certain embodiments, the pharmaceutical composition comprises IL-10 and a CD47 inhibitor. In certain embodiments, the pharmaceutical composition comprises IL-10 and an anti-CD47 antibody, an anti-SIRPα antibody, a SIRPα-Fc fusion protein, an anti-EGFR antibody, an anti-CD40 antibody, an anti-PD-L1 antibody, an anti-PD-L2 antibody, an anti-CD73 antibody, an anti-EpCAM antibody. In certain embodiments, the pharmaceutical composition comprises IL-10 and an anti-CD47 antibody. In certain embodiments, the pharmaceutical composition comprises IL-10 and magrolimab, TTI-621, TTI-622, AO-176, HX-009, AK117, AK112, CC90002, STI-6643, PF-07257876, IMC-002, CPO107, SRF231, TQB2928, IBI188, IBI322, IMM2902, BAT7104, TG-1801, SL-172154, BI 765063, or GS-0189.

In certain embodiments, the macrophage-directed immunotherapy and the cytokine are provided in an effective amount in the pharmaceutical composition. In certain embodiments, the effective amount is a therapeutically effective amount. In certain embodiments, a therapeutically effective amount is an amount effective for treating a cancer in a subject in need thereof. In certain embodiments, therapeutically effective amount is an amount effective for reducing, delaying, and/or preventing in a subject in need thereof the resistance of a cancer to a macrophage-directed immunotherapy and/or cytokine. In certain embodiments, the effective amount is a prophylactically effective amount (e.g., an amount effective for preventing a cancer in a subject in need thereof).

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile. In certain embodiments, the subject is with a cancer. In certain embodiments, the subject is with a cancer and has failed therapy of the cancer with a targeted agent (e.g., EGFR inhibitor) alone. In certain embodiments, the subject is with a cancer and has failed therapy of the cancer with a macrophage-directed immunotherapy alone.

In certain embodiments, the cell is in vitro. In certain embodiments, the cell is in vivo. In certain embodiments, the cell is a cell of a tissue or biological sample. In certain embodiments, the cell is a cancer cell.

Pharmaceutical compositions described herein can be prepared by any method known in the art of pharmacology. In general, such preparatory methods include bringing the macrophage-directed immunotherapy and/or cytokines described herein (i.e., the “active ingredients”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly (vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly (vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a macrophage-directed immunotherapy and/or cytokine described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the macrophage-directed immunotherapy and/or cytokine in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface-active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The macrophage-directed immunotherapy and/or cytokines provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The macrophage-directed immunotherapies, cytokines, and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the macrophage-directed immunotherapies, cytokines, and pharmaceutical compositions described herein are suitable for topical administration to the eye of a subject.

The exact amount (e.g., combined amount) of the macrophage-directed immunotherapy and cytokine required to achieve an effective amount will vary from subject to subject, depending, for example, on species, age, and general condition of a subject, severity of the side effects or disorder, identity of the particular macrophage-directed immunotherapy, identity of the particular cytokine, mode of administration, and the like. An effective amount may be included in a single dose (e.g., single oral dose) or multiple doses (e.g., multiple oral doses). Each dose is a combination of the macrophage-directed immunotherapy and the cytokine. For each dose, the macrophage-directed immunotherapy and the cytokine may be independently administered at the same time or administered separately at different times in any order. In certain embodiments, the duration between an administration of the macrophage-directed immunotherapy and an administration of the cytokine is about one hour, about two hours, about six hours, about twelve hours, about one day, about two days, about four days, or about one week, wherein the administration of the macrophage-directed immunotherapy and the administration of the cytokine are consecutive administrations. The macrophage-directed immunotherapy in each dose may be independently administered at the same time or administered separately at different times. The cytokine in each dose may also be independently administered at the same time or administered separately at different times. For example, in the following administrations: the cytokine in amount A, followed by the macrophage-directed immunotherapy in amount B1, and followed by the macrophage-directed immunotherapy in amount B2, the dose is the cytokine in amount A plus the macrophage-directed immunotherapy in amount (B1+B2). In certain embodiments, when multiple doses (e.g., multiple combinations of the macrophage-directed immunotherapy and the cytokine) are administered to a subject or applied to a biological sample, tissue, or cell, any about two doses of the multiple doses include different or substantially the same amounts of a macrophage-directed immunotherapy and/or cytokine described herein. In certain embodiments, when multiple doses are administered to a subject or applied to a biological sample, tissue, or cell, the frequency of administering the multiple doses to the subject or applying the multiple doses to the biological sample, tissue, or cell is about three doses a day, about two doses a day, about one dose a day, about one dose every other day, about one dose every third day, about one dose every week, about one dose every about two weeks, about one dose every about three weeks, or about one dose every about four weeks. In certain embodiments, the frequency of administering the multiple doses to the subject or applying the multiple doses to the biological sample, tissue, or cell is about one dose per day. In certain embodiments, the frequency of administering the multiple doses to the subject or applying the multiple doses to the biological sample, tissue, or cell is about two doses per day. In certain embodiments, the frequency of administering the multiple doses to the subject or applying the multiple doses to the biological sample, tissue, or cell is about three doses per day. In certain embodiments, when multiple doses are administered to a subject or applied to a biological sample, tissue, or cell, the duration between the first dose and last dose of the multiple doses is about one day, about two days, about four days, about one week, about two weeks, about three weeks, about one month, about two months, about three months, about four months, about six months, about nine months, about one year, about two years, about three years, about four years, about five years, about seven years, about ten years, fifteen years, twenty years, or the lifetime of the subject, tissue, or cell. In certain embodiments, the duration between the first dose and last dose of the multiple doses is about three months, about six months, or about one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 1 mg and 100 mg, between 30 mg and 100 mg, between 100 mg and 300 mg, between 1 mg and 1 g, between 300 mg and 1 g, between 1 mg and 10 g, or between 1 g and 10 g, inclusive, as the combined weight of a macrophage-directed immunotherapy and a cytokine described herein. In certain embodiments, a dose described herein includes independently between 1 mg and 3 mg, inclusive, as the combined weight of a macrophage-directed immunotherapy and a cytokine described herein. In certain embodiments, a dose described herein includes independently between 3 mg and 10 mg, inclusive, as the combined weight of a macrophage-directed immunotherapy and a cytokine described herein. In certain embodiments, a dose described herein includes independently between 10 mg and 30 mg, inclusive, as the combined weight of a macrophage-directed immunotherapy and a cytokine described herein. In certain embodiments, a dose described herein includes independently between 30 mg and 100 mg, inclusive, as the combined weight of a macrophage-directed immunotherapy and a cytokine described herein.

Doses and dose ranges described herein provide guidance for the administration of provided pharmaceutical compositions to an adult (e.g., an adult whose body weight is 70 kg). The amount to be administered to, for example, a child or an adolescent can be determined by a medical practitioner or person skilled in the art and can be lower or the same as that administered to an adult.

The combinations of the macrophage-directed immunotherapy and the cytokine are expected to be synergistic in treating and/or preventing in the subject the cancers, in reducing, delaying, and/or preventing in the subject the resistance of cancers to a macrophage-directed immunotherapy and/or cytokine, in inhibiting the proliferation of the cell, and/or reducing, delaying, and/or preventing the resistance of the cell to a macrophage-directed immunotherapy and/or cytokine, compared to the macrophage-directed immunotherapy alone or the cytokine alone. To result in the same effect in treating and/or preventing in the subject the cancers, in reducing, delaying, and/or preventing in the subject the resistance of cancers to a macrophage-directed immunotherapy and/or cytokine, in inhibiting the proliferation of the cell, and/or reducing, delaying, and/or preventing the resistance of the cell to a macrophage-directed immunotherapy and/or cytokine, a dose of a combination of the macrophage-directed immunotherapy and the cytokine may be lower than (e.g., lower than 0.1%, lower than 1%, lower than 10%, or lower than 30%) a dose of the macrophage-directed immunotherapy alone and lower than a dose of the cytokine alone. To result in the same effect in treating and/or preventing in the subject the cancers, in reducing, delaying, and/or preventing in the subject the resistance of cancers to a macrophage-directed immunotherapy and/or cytokine, in inhibiting the proliferation of the cell, and/or reducing, delaying, and/or preventing the resistance of the cell to a macrophage-directed immunotherapy and/or cytokine, the frequency of multiple doses of a combination of the macrophage-directed immunotherapy and the cytokine may be lower than (e.g., lower than 0.1%, lower than 1%, lower than 10%, or lower than 30%) the frequency of multiple doses of the macrophage-directed immunotherapy alone and lower than a dose of the cytokine alone. To result in the same effect in treating and/or preventing in the subject the cancers, in reducing, delaying, and/or preventing in the subject the resistance of cancers to a macrophage-directed immunotherapy and/or cytokine, in inhibiting the proliferation of the cell, and/or reducing, delaying, and/or preventing the resistance of the cell to a macrophage-directed immunotherapy and/or cytokine, the total amount of multiple doses of a combination of the macrophage-directed immunotherapy and the cytokine may be lower than (e.g., lower than 0.1%, lower than 1%, lower than 10%, or lower than 30%) the total amount of multiple doses of the macrophage-directed immunotherapy alone and lower than a dose of the cytokine alone.

A macrophage-directed immunotherapy, cytokine, or composition, as described herein, can be administered in combination with one or more additional pharmaceutical agents (e.g., therapeutically and/or prophylactically active agents). The macrophage-directed immunotherapy, cytokine, or composition can be administered in combination with additional pharmaceutical agents that improve their activity (e.g., activity (e.g., potency and/or efficacy) in treating a cancer in a subject in need thereof, in preventing a cancer in a subject in need thereof, in reducing, delaying, and/or preventing in a subject in need thereof the resistance of cancers to a macrophage-directed immunotherapy and/or cytokine, in inhibiting the proliferation of a cell, in reducing, delaying, and/or preventing the resistance of a cell to a macrophage-directed immunotherapy and/or cytokine), improve bioavailability, improve safety, reduce drug resistance, reduce and/or modify metabolism, inhibit excretion, and/or modify distribution in a subject, biological sample, tissue, or cell. It will also be appreciated that the therapy employed may achieve a desired effect for the same disorder, and/or it may achieve different effects. In certain embodiments, a pharmaceutical composition described herein including (1) a macrophage-directed immunotherapy and a cytokine described herein, and (2) an additional pharmaceutical agent shows a synergistic effect, compared with a pharmaceutical composition including one of (1) and (2), but not both (1) and (2).

The macrophage-directed immunotherapy, cytokine, or composition can be independently administered concurrently with, prior to, or subsequent to one or more additional pharmaceutical agents. In certain embodiments, the additional pharmaceutical agents and the macrophage-directed immunotherapy are not the same, and the additional pharmaceutical agents and the cytokine are not the same. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease). Each additional pharmaceutical agent may be administered at a dose and/or on a time schedule determined for that pharmaceutical agent. The additional pharmaceutical agents may also be administered together with each other and/or with the macrophage-directed immunotherapy, cytokine, or composition described herein at the same time or administered separately at different times. The particular combination to employ in a regimen will take into account compatibility of the macrophage-directed immunotherapy and/or cytokine described herein with the additional pharmaceutical agent(s), and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, pain-relieving agents, and combinations thereof. In certain embodiments, the additional pharmaceutical agent is an anti-proliferative agent (e.g., anti-cancer agent, cytotoxic agent).

Also encompassed by the disclosure are kits (e.g., pharmaceutical packs). The kits provided may comprise a macrophage-directed immunotherapy and a cytokine described herein, a bifunctional compound as described herein, or a pharmaceutical composition described herein. The kits may comprise a macrophage-directed immunotherapy and a cytokine in a first container. The kits may comprise a macrophage-directed immunotherapy in a first container and a cytokine in a second container. The kits may comprise a pharmaceutical composition in a first container. In some embodiments, the kits further include a third container comprising a pharmaceutical excipient for dilution or suspension of the macrophage-directed immunotherapy, cytokine, and/or pharmaceutical composition. In some embodiments, the macrophage-directed immunotherapy, cytokine, or pharmaceutical composition provided in the first container, optionally the second container, and optionally the third container are combined to form one unit dosage form. Each of the first container, second container, and third container may independently be a vial, ampule, bottle, syringe, and/or dispenser package, or other suitable container. In certain embodiments, the kits are useful for treating a cancer (e.g., cancer that is resistant to a macrophage-directed immunotherapy and/or cytokine) in a subject in need thereof. In certain embodiments, the kits are useful for preventing a cancer (e.g., cancer that is resistant to a macrophage-directed immunotherapy and/or cytokine) in a subject in need thereof. In certain embodiments, the kits are useful for reducing, delaying, and/or preventing in a subject in need thereof the resistance of a cancer to a macrophage-directed immunotherapy and/or cytokine. In certain embodiments, the kits are useful in inhibiting the proliferation of a cell. In certain embodiments, the kits are useful in reducing, delaying, and/or preventing the resistance of a cell to a macrophage-directed immunotherapy and/or cytokine. In certain embodiments, a kit described herein further includes instructions for using the macrophage-directed immunotherapy and cytokine included in the kit, or for using the pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a cancer (e.g., cancer that is resistant to a macrophage-directed immunotherapy and/or cytokine) in a subject in need thereof. In certain embodiments, the kits and instructions provide for preventing a cancer (e.g., cancer that is resistant to a macrophage-directed immunotherapy and/or cytokine) in a subject in need thereof. In certain embodiments, the kits and instructions provide for reducing, delaying, and/or preventing in a subject in need thereof the resistance of a cancer to a macrophage-directed immunotherapy and/or cytokine. In certain embodiments, the kits and instructions provide for inhibiting the proliferation of a cell. In certain embodiments, the kits and instructions provide for reducing, delaying, and/or preventing the resistance of a cell to a macrophage-directed immunotherapy and/or cytokine. A kit described herein may include one or more additional pharmaceutical agents described herein as a separate composition.

### EXAMPLES

In order that the present disclosure may be more fully understood, the following examples are set forth. The examples described in this application are offered to illustrate the methods, macrophage-directed immunotherapies, cytokines, and pharmaceutical compositions provided herein and are not to be construed in any way as limiting their scope.

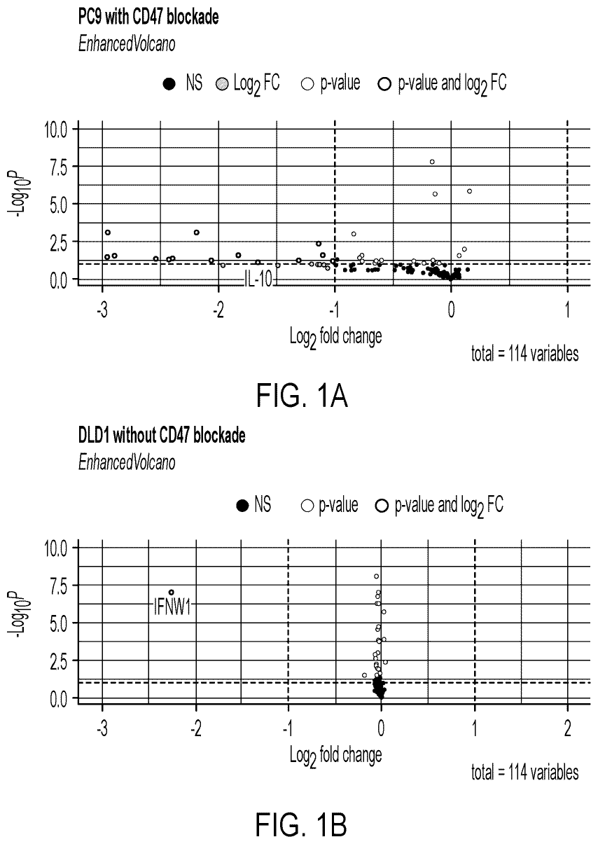

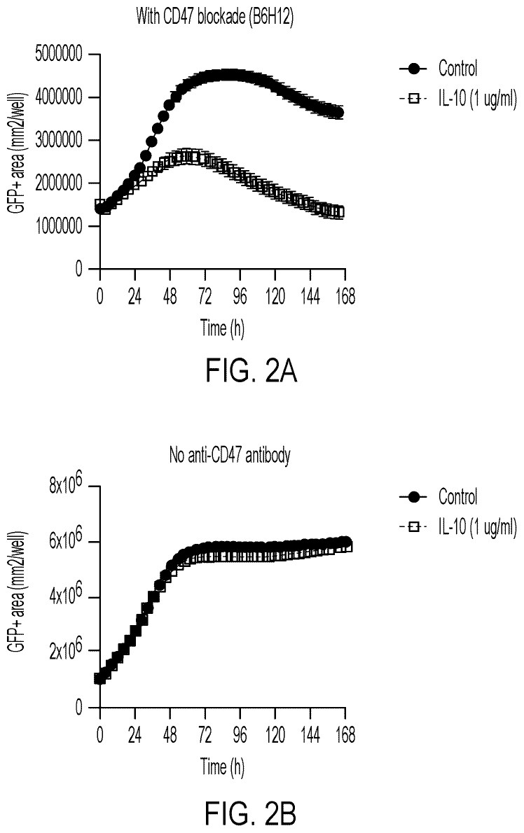

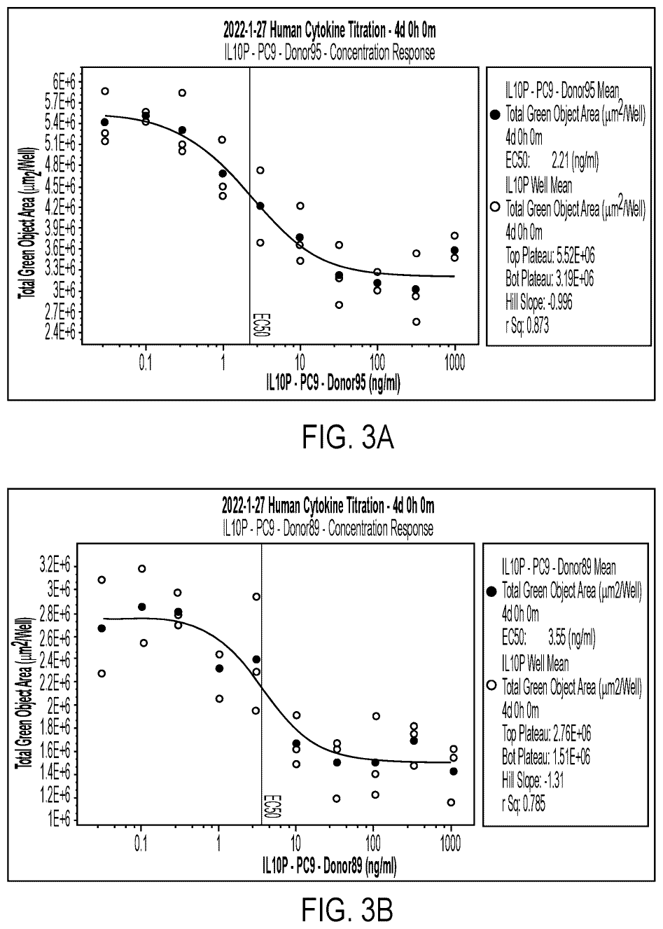

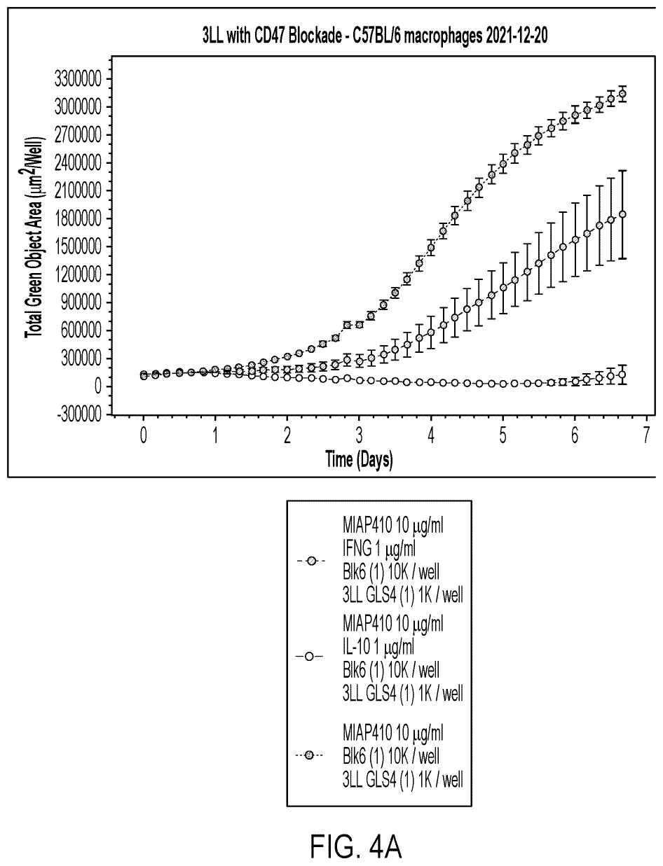

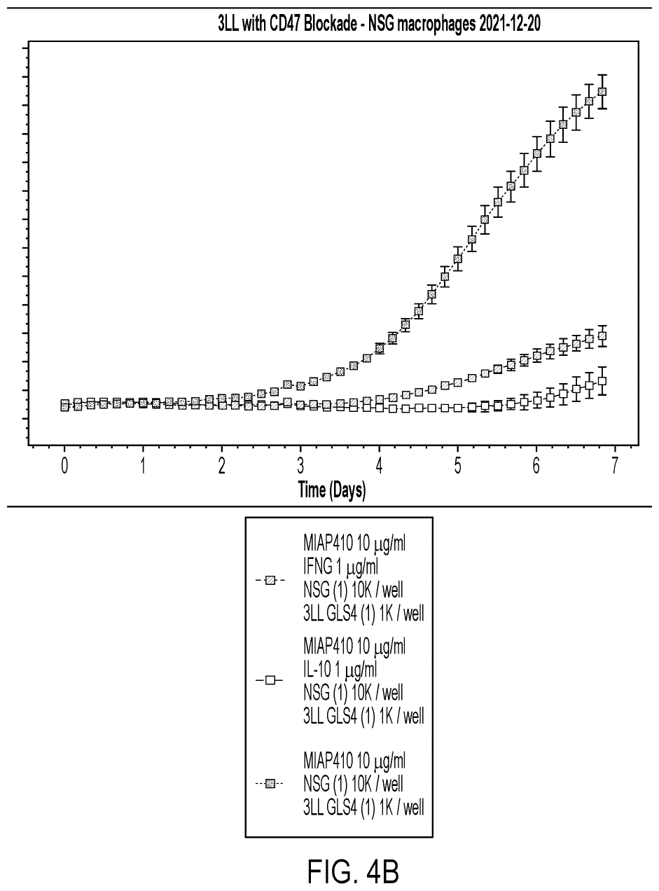

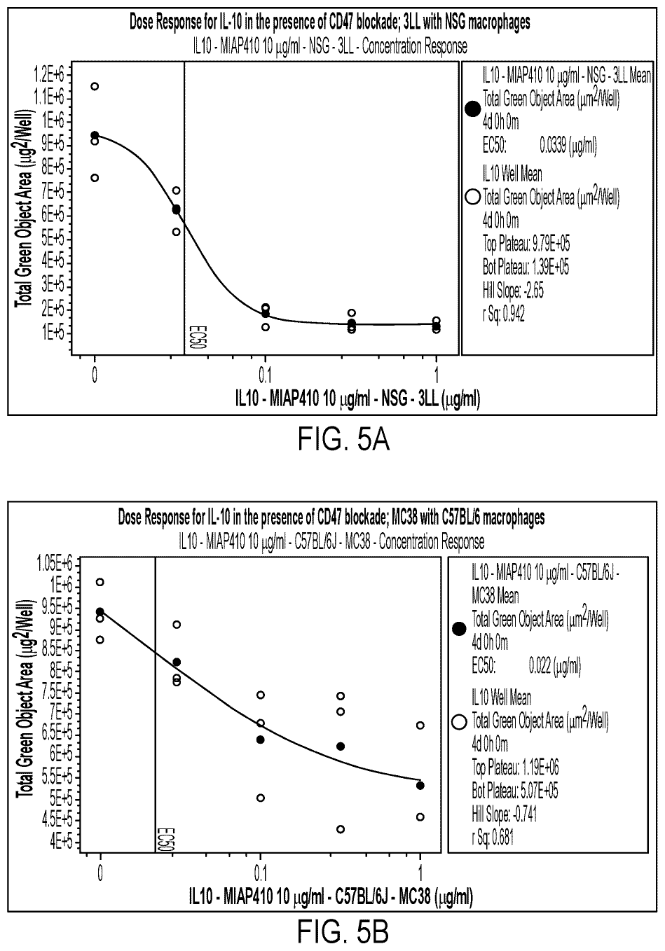

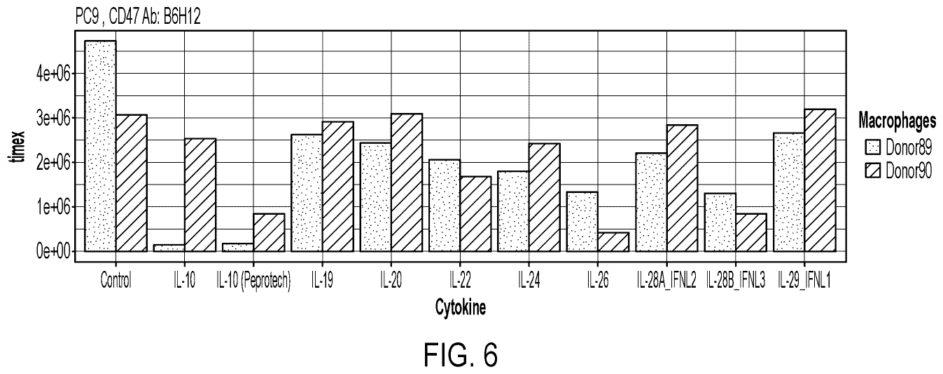

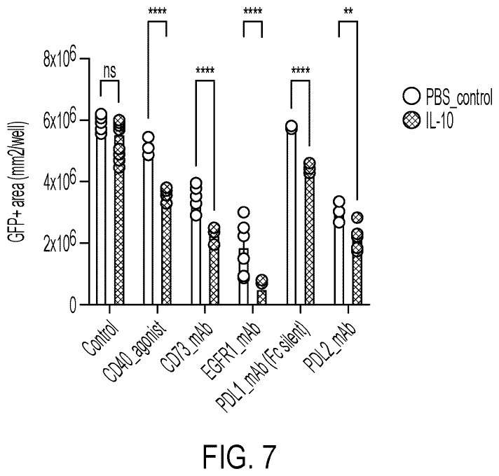

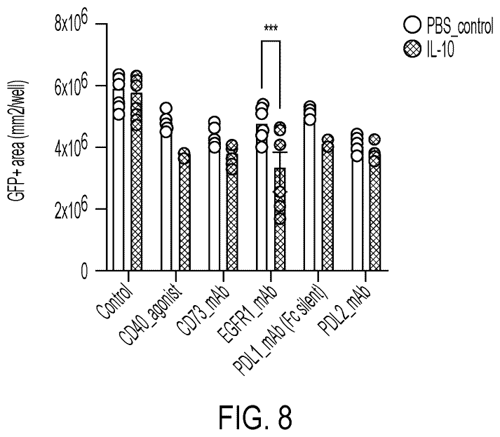

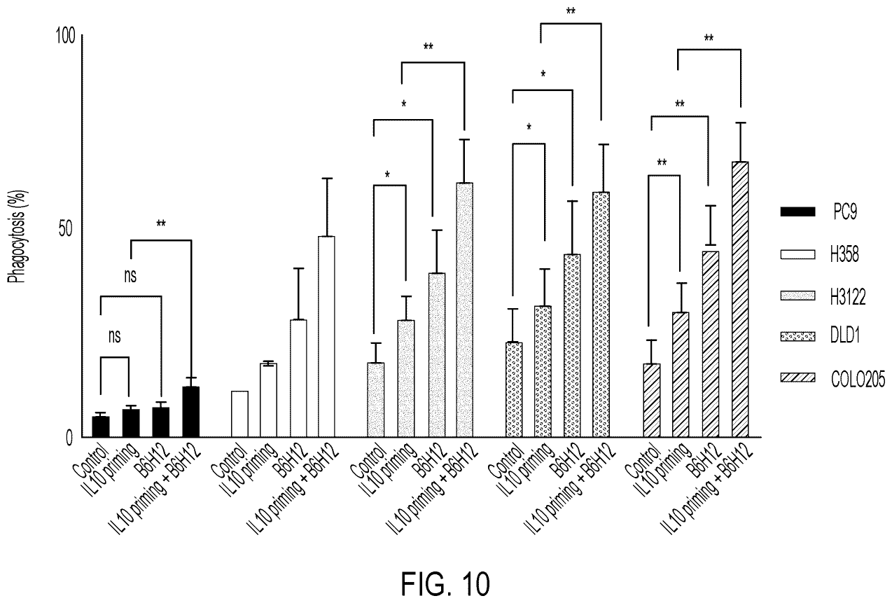

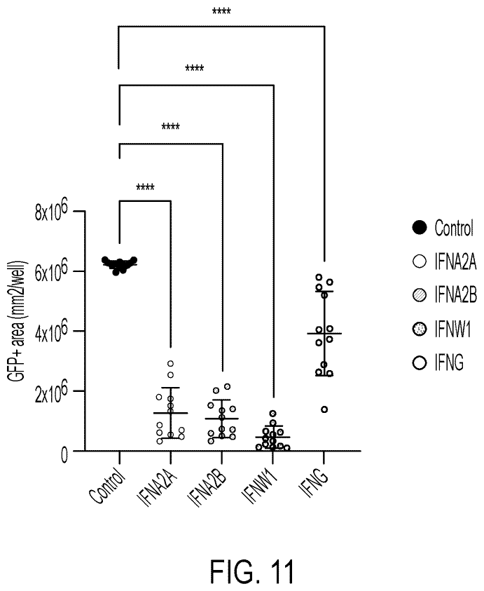

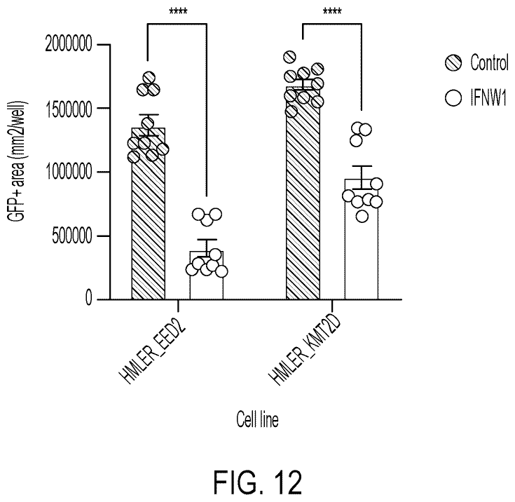

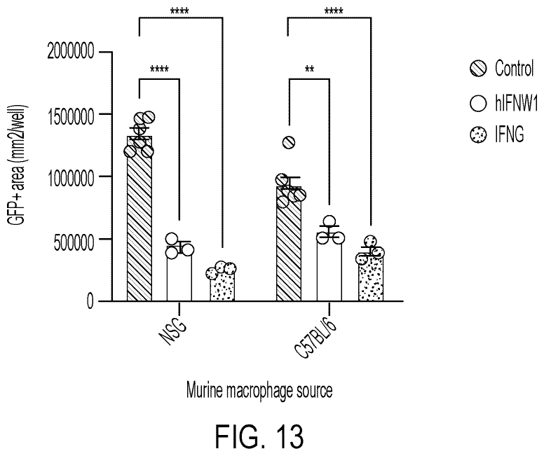

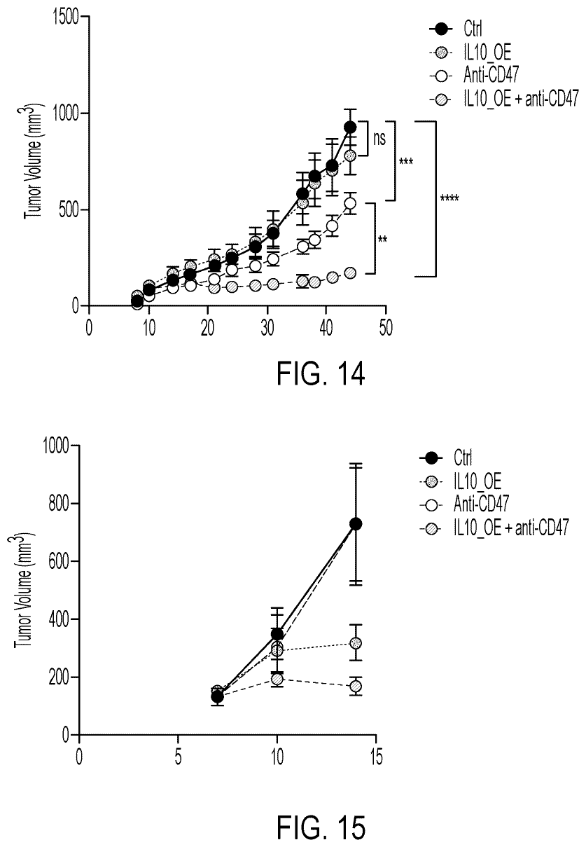

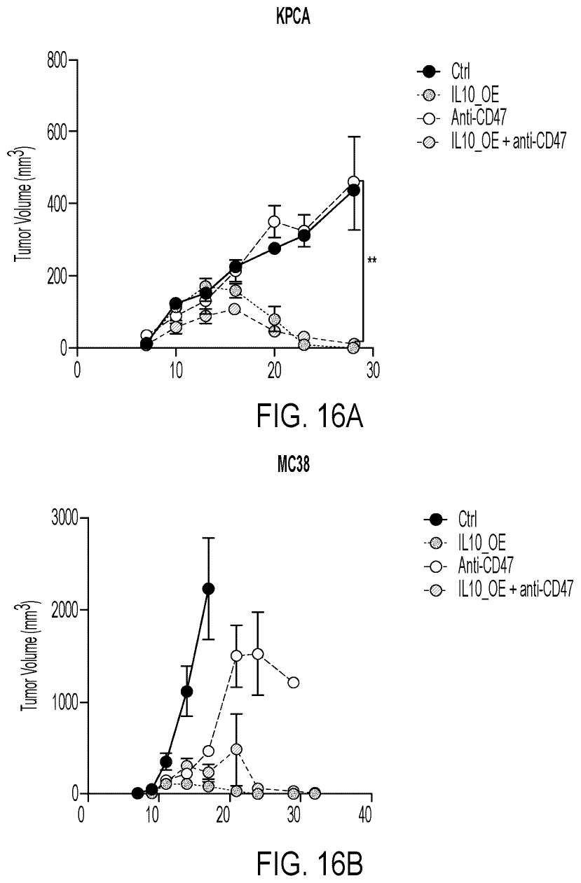

Experiments demonstrating and enabling the practice of the subject matter of the present disclosure are described thoroughly in FIGS. 1-16. IL-10 was purchased from Peprotech (www.peprotech.com/en/recombinant-human-il-10-2).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating a proliferative disease in a subject in need thereof, the method comprising administering a macrophage-directed immunotherapy and a cytokine, or a modification, fragment, or variant thereof.

2. The method of claim 1, wherein the macrophage-directed immunotherapy is an immunotherapeutic agent.

3. The method of claim 1 or 2, wherein the macrophage-directed immunotherapy is a macrophage immune checkpoint inhibitor.

4. The method of any of claims 1-3, wherein the macrophage-directed immunotherapy is a CD47 inhibitor, a SIRPα inhibitor, an EGFR inhibitor, an MHC I inhibitor, a CD24 inhibitor, a CALR inhibitor, a CD40 agonist, a PD-L1 inhibitor, an APMAP inhibitor, a GPR84 inhibitor, a VCAM1 inhibitor, a CD11b inhibitor, a SIGLEC-10 inhibitor, a PD-L2 inhibitor, a PD-1 inhibitor, a CD73 inhibitor, an EpCAM inhibitor, a Galectin-9 inhibitor, a CD14 inhibitor, a CD80 inhibitor, a CD86 inhibitor, a SIRPb inhibitor, a SIRPg inhibitor, a SLAMF7 inhibitor, a MARCO inhibitor, an AXL inhibitor, a CLEVER-1 inhibitor, an ILT4 inhibitor, a TIM-3 inhibitor, a TIM-4 inhibitor, an LRP-1 inhibitor, a calreticulin inhibitor, a TREM1 inhibitor, a TREM2 inhibitor, a GD2 inhibitor, an FcgRI inhibitor, an FcgRIIa inhibitor, an FcgRIIb inhibitor, an FcgRIII inhibitor, a MUC1 inhibitor, a CD44 inhibitor, a CD63 inhibitor, a CD36 inhibitor, a CD84 inhibitor, a CD164 inhibitor, a CD82 inhibitor, a CD18 inhibitor, a SIGLEC-7 inhibitor, a CD166 inhibitor, a CD39 inhibitor, a CD46 inhibitor, an LILRA1 inhibitor, an LILRA2 inhibitor, an LILRA3 inhibitor, an LILRA4 inhibitor, an LILRB1 inhibitor, an LILRB2 inhibitor, an LILRB3 inhibitor, an LILRB4 inhibitor, an LILRB5 inhibitor, a CD85b inhibitor, a CD85m inhibitor, a CD85f inhibitor, a CD276 inhibitor, a CD88 inhibitor, a CD99 inhibitor, a PILRa inhibitor, a Siglec-9 inhibitor, a CD206 inhibitor, a CD163 inhibitor, a CD84 inhibitor, a C3aR inhibitor, or a CLEC12A inhibitor.

5. The method of any of claims 1-4, wherein the macrophage-directed immunotherapy is a CD47 inhibitor, a SIRPα inhibitor, an EGFR inhibitor, a CD40 agonist, a PD-L1 inhibitor, a PD-L2 inhibitor, a CD73 inhibitor, or an EpCAM inhibitor.

6. The method of any of claims 1-5, wherein the macrophage-directed immunotherapy is a biologic.

7. The method of any of claims 1-6, wherein the macrophage-directed immunotherapy is an antibody or antibody fragment.

8. The method of any of claims 1-7, wherein the macrophage-directed immunotherapy is an anti-CD47 antibody, an anti-SIRPα antibody, a SIRPα-Fc fusion protein, an anti-EGFR antibody, an anti-MHC I antibody, an anti-CD24 antibody, an anti-CALR antibody, an anti-CD40 antibody, an anti-PD-L1 antibody, an anti-APMAP antibody, an anti-GPR84 antibody, an anti-VCAM1 antibody, an anti-CD11b antibody, an anti-SIGLEC-10 antibody, an anti-PD-L2 antibody, an anti-PD-1 antibody, an anti-CD73 antibody, an anti-EpCAM antibody, an anti-Galectin-9 antibody, an anti-CD14 antibody, an anti-CD80 antibody, an anti-CD86 antibody, an anti-SIRPb antibody, an anti-SIRPg antibody, an anti-SLAMF7 antibody, an anti-MARCO antibody, an anti-AXL antibody, an anti-CLEVER-1 antibody, an anti-ILT4 antibody, an anti-TIM-3 antibody, an anti-TIM-4 antibody, an anti-LRP-1 antibody, an anti-calreticulin antibody, an anti-TREM1 antibody, an anti-TREM2 antibody, an anti-GD2 antibody, an anti-FcgRI antibody, an anti-FcgRIIa antibody, an anti-FcgRIIb antibody, an anti-FcgRIII antibody, an anti-MUC1 antibody, an anti-CD44 antibody, an anti-CD63 antibody, an anti-CD36 antibody, an anti-CD84 antibody, an anti-CD164 antibody, an anti-CD82 antibody, an anti-CD18 antibody, an anti-SIGLEC-7 antibody, an anti-CD166 antibody, an anti-CD39 antibody, an anti-CD46 antibody, an anti-LILRA1 antibody, an anti-LILRA2 antibody, an anti-LILRA3 antibody, an anti-LILRA4 antibody, an anti-LILRB1 antibody, an anti-LILRB2 antibody, an anti-LILRB3 antibody, an anti-LILRB4 antibody, an anti-LILRB5 antibody, an anti-CD85b antibody, an anti-CD85m antibody, an anti-CD85f antibody, an anti-CD276 antibody, an anti-CD88 antibody, an anti-CD99 antibody, an anti-PILRa antibody, an anti-Siglec-9 antibody, an anti-CD206 antibody, an anti-CD163 antibody, an anti-CD84 antibody, an anti-C3aR antibody, or an anti-CLEC12A antibody.

9. The method of any of claims 1-8, wherein the macrophage-directed immunotherapy is an anti-CD47 antibody, an anti-SIRPα antibody, a SIRPα-Fc fusion protein, an anti-EGFR antibody, an anti-CD40 antibody, an anti-PD-L1 antibody, an anti-PD-L2 antibody, an anti-CD73 antibody, or an anti-EpCAM antibody.

10. The method of any of claims 1-9, wherein the macrophage-directed immunotherapy is an anti-CD47 antibody.

11. The method of any of claims 1-10, wherein the macrophage-directed immunotherapy is magrolimab, TTI-621, TTI-622, AO-176, HX-009, AK117, AK112, CC90002, STI-6643, PF-07257876, TQB2928, IMC-002, CPO107, SRF231, IBI188, IBI322, IMM2902, BAT7104, TG-1801, SL-172154, BI 765063, or GS-0189.

12. The method of any of claims 1-11, wherein the cytokine is an anti-inflammatory cytokine or a modification, fragment, or variant thereof.

13. The method of any of claims 1-12, wherein the cytokine is a class II cytokine or a modification, fragment, or variant thereof.

14. The method of any of claims 1-13, wherein the cytokine is interleukin-10 (IL-10), interleukin-19 (IL-19), interleukin-20 (IL-20), interleukin-22 (IL-22), interleukin-24 (IL-24), interleukin-26 (IL-26), interleukin-28A (IL-28A), interleukin-28B (IL-28B), interleukin-29 (IL-29), a type-I interferon, a type-II interferon, or a modification, fragment, or variant thereof.

15. The method of any of claims 1-14, wherein the cytokine is interleukin-10 (IL-10), interleukin-19 (IL-19), interleukin-20 (IL-20), interleukin-22 (IL-22), interleukin-24 (IL-24), interleukin-26 (IL-26), interleukin-28A (IL-28A), interleukin-28B (IL-28B), or interleukin-29 (IL-29), or a modification, fragment, or variant thereof.

16. The method of any of claims 1-15, wherein the cytokine is interleukin-10 (IL-10), or a modification, fragment, or variant thereof.

17. The method of any of claims 1-16, wherein the cytokine is a variant of interleukin-10 (IL-10) having at least 80%, 85%, 90%, 95%, 97%, 98%, 99%, or 99.5% sequence identity to the amino acid sequence of IL-10.

18. The method of any of claims 1-15, wherein the cytokine is a modification of interleukin-10 (IL-10).

19. The method of any of claim 1-15 or 18, wherein the cytokine is pegilodecakin.

20. The method of any of claims 1-14, wherein the cytokine is a type-I interferon or a type-II interferon, or a modification, fragment, or variant thereof.

21. The method of any of claim 1-14 or 20, wherein the cytokine is interferon alpha (IFN-α), interferon gamma (IFN-γ), or interferon omega (IFN-ω), or a modification, fragment, or variant thereof.

22. The method of any of claim 1-14, 20, or 21 wherein the cytokine is interferon omega (IFN-ω), or a modification, fragment, or variant thereof.

23. The method of any of claims 1-22, wherein the proliferative disease is cancer.

24. The method of claim 23, wherein the cancer is bladder cancer, cervical cancer, dermatofibrosarcoma protuberans, endocrine tumors, neuroendocrine tumors, neuroblastoma, anaplastic large cell lymphoma, glioblastoma multiforme, bile duct cancer, stomach cancer, colon cancer, rectal cancer, melanoma, colorectal cancer, brain cancer, head and neck cancer, thyroid cancer, soft tissue cancer, lung cancer, colon cancer, kidney cancer, liver cancer, gastric cancer, gastrointestinal stromal tumor, giant cell tumor, esophageal cancer, gastroesophageal cancer, breast cancer, ovarian cancer, prostate cancer, endometrial cancer, pancreatic cancer, leukemia, lymphoma, multiple myeloma, colon adenocarcinoma, lung adenocarcinoma, cutaneous melanoma, gastrointestinal cancer, anal cancer, glioblastoma, epithelian tumors of the head and neck, laryngeal cancer, oral cancer, myelodysplastic disorders, myeloproliferative disorders, ovarian epithelial cancer, fallopian tube cancer, primary peritoneal cancer, plexiform neurofibroma, skin cancer, soft tissue sarcoma, solid tumors with an NTRK gene fusion, or systemic mastocytosis.

25. The method of claim 23 or 24, wherein the cancer is colon cancer.

26. The method of claim 23 or 24, wherein the cancer is lung cancer.

27. The method of any of claim 23, 24, or 26, wherein the cancer is non-small cell lung cancer.

28. The method of any of claims 1-27, wherein the combination of the macrophage-directed immunotherapy and the cytokine are synergistic in treating the proliferative disease, compared to treatment with the macrophage-directed immunotherapy alone and/or treatment with the cytokine alone.

29. The method of any of claims 1-28, wherein the treatment results in an increase in phagocytosis of cancer cells compared to treatment with the macrophage-directed immunotherapy alone.

30. The method of any of claims 1-29, wherein the treatment results in an increase of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% in phagocytosis of cancer cells compared to treatment with the macrophage-directed immunotherapy alone.

31. The method of claim 29 or 30, wherein the cancer cells are colon cancer cells or non-small cell lung cancer cells.

32. The method of any of claims 1-31, wherein the subject is a mammal.

33. The method of any of claims 1-32, wherein the subject is a human.

34. A pharmaceutical composition comprising a macrophage-directed immunotherapy and a cytokine, or a modification, fragment, or variant thereof; and optionally a pharmaceutically acceptable excipient.

35. The pharmaceutical composition of claim 34, wherein the macrophage-directed immunotherapy is an immunotherapeutic agent.

36. The pharmaceutical composition of claim 34 or 35, wherein the macrophage-directed immunotherapy is a macrophage immune checkpoint inhibitor.

37. The pharmaceutical composition of any of claims 34-36, wherein the macrophage-directed immunotherapy is a CD47 inhibitor, a SIRPα inhibitor, an EGFR inhibitor, an MHC I inhibitor, a CD24 inhibitor, a CALR inhibitor, a CD40 agonist, a PD-L1 inhibitor, an APMAP inhibitor, a GPR84 inhibitor, a VCAM1 inhibitor, a CD11b inhibitor, a SIGLEC-10 inhibitor, a PD-L2 inhibitor, a PD-1 inhibitor, a CD73 inhibitor, an EpCAM inhibitor, a Galectin-9 inhibitor, a CD14 inhibitor, a CD80 inhibitor, a CD86 inhibitor, a SIRPb inhibitor, a SIRPg inhibitor, a SLAMF7 inhibitor, a MARCO inhibitor, an AXL inhibitor, a CLEVER-1 inhibitor, an ILT4 inhibitor, a TIM-3 inhibitor, a TIM-4 inhibitor, an LRP-1 inhibitor, a calreticulin inhibitor, a TREM1 inhibitor, a TREM2 inhibitor, a GD2 inhibitor, an FcgRI inhibitor, an FcgRIIa inhibitor, an FcgRIIb inhibitor, an FcgRIII inhibitor, a MUC1 inhibitor, a CD44 inhibitor, a CD63 inhibitor, a CD36 inhibitor, a CD84 inhibitor, a CD164 inhibitor, a CD82 inhibitor, a CD18 inhibitor, a SIGLEC-7 inhibitor, a CD166 inhibitor, a CD39 inhibitor, a CD46 inhibitor, an LILRA1 inhibitor, an LILRA2 inhibitor, an LILRA3 inhibitor, an LILRA4 inhibitor, an LILRB1 inhibitor, an LILRB2 inhibitor, an LILRB3 inhibitor, an LILRB4 inhibitor, an LILRB5 inhibitor, a CD85b inhibitor, a CD85m inhibitor, a CD85f inhibitor, a CD276 inhibitor, a CD88 inhibitor, a CD99 inhibitor, a PILRa inhibitor, a Siglec-9 inhibitor, a CD206 inhibitor, a CD163 inhibitor, a CD84 inhibitor, a C3aR inhibitor, or a CLEC12A inhibitor.

38. The pharmaceutical composition of any of claims 34-37, wherein the macrophage-directed immunotherapy is a CD47 inhibitor, a SIRPα inhibitor, an EGFR inhibitor, a CD40 agonist, a PD-L1 inhibitor, a PD-L2 inhibitor, a CD73 inhibitor, or an EpCAM inhibitor.

39. The pharmaceutical composition of any of claims 34-38, wherein the macrophage-directed immunotherapy is a biologic.

40. The pharmaceutical composition of any of claims 34-39, wherein the macrophage-directed immunotherapy is an antibody or antibody fragment.

41. The pharmaceutical composition of any of claims 34-40, wherein the macrophage-directed immunotherapy is an anti-CD47 antibody, an anti-SIRPα antibody, a SIRPα-Fc fusion protein, an anti-EGFR antibody, an anti-MHC I antibody, an anti-CD24 antibody, an anti-CALR antibody, an anti-CD40 antibody, an anti-PD-L1 antibody, an anti-APMAP antibody, an anti-GPR84 antibody, an anti-VCAM1 antibody, an anti-CD11b antibody, an anti-SIGLEC-10 antibody, an anti-PD-L2 antibody, an anti-PD-1 antibody, an anti-CD73 antibody, an anti-EpCAM antibody, an anti-Galectin-9 antibody, an anti-CD14 antibody, an anti-CD80 antibody, an anti-CD86 antibody, an anti-SIRPb antibody, an anti-SIRPg antibody, an anti-SLAMF7 antibody, an anti-MARCO antibody, an anti-AXL antibody, an anti-CLEVER-1 antibody, an anti-ILT4 antibody, an anti-TIM-3 antibody, an anti-TIM-4 antibody, an anti-LRP-1 antibody, an anti-calreticulin antibody, an anti-TREM1 antibody, an anti-TREM2 antibody, an anti-GD2 antibody, an anti-FcgRI antibody, an anti-FcgRIIa antibody, an anti-FcgRIIb antibody, an anti-FcgRIII antibody, an anti-MUC1 antibody, an anti-CD44 antibody, an anti-CD63 antibody, an anti-CD36 antibody, an anti-CD84 antibody, an anti-CD164 antibody, an anti-CD82 antibody, an anti-CD18 antibody, an anti-SIGLEC-7 antibody, an anti-CD166 antibody, an anti-CD39 antibody, an anti-CD46 antibody, an anti-LILRA1 antibody, an anti-LILRA2 antibody, an anti-LILRA3 antibody, an anti-LILRA4 antibody, an anti-LILRB1 antibody, an anti-LILRB2 antibody, an anti-LILRB3 antibody, an anti-LILRB4 antibody, an anti-LILRB5 antibody, an anti-CD85b antibody, an anti-CD85m antibody, an anti-CD85f antibody, an anti-CD276 antibody, an anti-CD88 antibody, an anti-CD99 antibody, an anti-PILRa antibody, an anti-Siglec-9 antibody, an anti-CD206 antibody, an anti-CD163 antibody, an anti-CD84 antibody, an anti-C3aR antibody, or an anti-CLEC12A antibody.

42. The pharmaceutical composition of any of claims 34-41, wherein the macrophage-directed immunotherapy is an anti-CD47 antibody, an anti-SIRPα antibody, a SIRPα-Fc fusion protein, an anti-EGFR antibody, an anti-CD40 antibody, an anti-PD-L1 antibody, an anti-PD-L2 antibody, an anti-CD73 antibody, or an anti-EpCAM antibody.

43. The pharmaceutical composition of any of claims 34-42, wherein the macrophage-directed immunotherapy is an anti-CD47 antibody.

44. The pharmaceutical composition of any of claims 34-43, wherein the macrophage-directed immunotherapy is magrolimab, TTI-621, TTI-622, AO-176, HX-009, AK117, AK112, CC90002, STI-6643, PF-07257876, TQB2928, IMC-002, CPO107, SRF231, IBI188, IBI322, IMM2902, BAT7104, TG-1801, SL-172154, BI 765063, or GS-0189.

45. The pharmaceutical composition of any of claims 34-44, wherein the cytokine is an anti-inflammatory cytokine, or a modification, fragment, or variant thereof.

46. The pharmaceutical composition of any of claims 34-45, wherein the cytokine is a class II cytokine, or a fragment or variant thereof.

47. The pharmaceutical composition of any of claims 34-46, wherein the cytokine is interleukin-10 (IL-10), interleukin-19 (IL-19), interleukin-20 (IL-20), interleukin-22 (IL-22), interleukin-24 (IL-24), interleukin-26 (IL-26), interleukin-28A (IL-28A), interleukin-28B (IL-28B), interleukin-29 (IL-29), a type-I interferon, a type-II interferon, or a modification, fragment, or variant thereof.

48. The pharmaceutical composition of any of claims 34-47, wherein the cytokine is interleukin-10 (IL-10), interleukin-19 (IL-19), interleukin-20 (IL-20), interleukin-22 (IL-22), interleukin-24 (IL-24), interleukin-26 (IL-26), interleukin-28A (IL-28A), interleukin-28B (IL-28B), or interleukin-29 (IL-29), or a modification, fragment, or variant thereof.

49. The pharmaceutical composition of any of claims 34-48, wherein the cytokine is interleukin-10 (IL-10), or a modification, fragment, or variant thereof.

50. The pharmaceutical composition of any of claims 34-49, wherein the cytokine is a variant of interleukin-10 (IL-10) having at least 80%, 85%, 90%, 95%, 97%, 98%, 99%, or 99.5% sequence identity to the amino acid sequence of IL-10.

51. The pharmaceutical composition of any of claims 34-49, wherein the cytokine is a modification of interleukin-10 (IL-10).

52. pharmaceutical composition of any of claim 34-49 or 51, wherein the cytokine is pegilodecakin.

53. The pharmaceutical composition of any of claims 34-47, wherein the cytokine is a type-I interferon or a type-II interferon, or a modification, fragment, or variant thereof.

54. The pharmaceutical composition of any of claim 34-47 or 53, wherein the cytokine is interferon alpha (IFN-α), interferon gamma (IFN-γ), or interferon omega (IFN-ω), or a modification, fragment, or variant thereof.

55. The pharmaceutical composition of any of claim 34-47, 53, or 54, wherein the cytokine is interferon omega (IFN-ω), or a modification, fragment, or variant thereof.

56. The pharmaceutical composition of any of claims 34-49, wherein the macrophage-directed immunotherapy is an anti-CD47 antibody; and the cytokine is interleukin-10 (IL-10), or a modification, fragment, or variant thereof.

57. The pharmaceutical composition of any of claims 34-49, wherein the macrophage-directed immunotherapy is an anti-CD47 antibody; and the cytokine is pegilodecakin.

58. A bifunctional compound, or a pharmaceutically acceptable salt thereof, comprising a macrophage-directed immunotherapy and a cytokine, or a modification, fragment, or variant thereof.

59. The bifunctional compound of claim 58, or a pharmaceutically acceptable salt thereof, wherein the bifunctional compound is a fusion protein.

60. The bifunctional compound of claim 58, or a pharmaceutically acceptable salt thereof, wherein the macrophage-directed immunotherapy and cytokine, or modification, fragment, or variant thereof, are attached through a linker.

61. The bifunctional compound of claim 60, or a pharmaceutically acceptable salt thereof, wherein the linker is a covalent linker.

62. The bifunctional compound of claim 60 or 61, or a pharmaceutically acceptable salt thereof, wherein the linker is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; and each R^{A} is independently hydrogen, a protecting group, or substituted or unsubstituted alkyl.

63. A pharmaceutical composition comprising the bifunctional compound of any of claims 58-62, or a pharmaceutically acceptable salt thereof; and optionally a pharmaceutically acceptable excipient.

64. A method of treating a proliferative disease in a subject in need thereof, the method comprising administering the bifunctional compound of any of claims 58-62, or a pharmaceutically acceptable salt thereof.

65. A kit comprising a macrophage-directed immunotherapy and a cytokine; and instructions for using the kit.
